(19) United States
(12) Patent Application Publication
(10) Pub. No.: US 2005/0233461 A1
(43) Pub. Date: Oct. 20, 2005

(54) High-throughput methods and systems for screening of compounds to treat/prevent kidney disorders

(76) Inventors: Douglas Levinson, Sherborn, MA (US); Kurt Amsler, Southborough, MA (US); Matthew Peterson, Hopkinton, MA (US)

Correspondence Address: TRANSFORM PHARMACEUTICALS, INC., 29 HARTWELL AVENUE, LEXINGTON, MA, 02421, US

(21) Appl. No.: 10/517,044

(22) PCT Filed: Jun. 4, 2003
(86) PCT No.: PCT/US03/17574

§ 371 Date: Dec. 3, 2004

Related U.S. Application Data
(60) Provisional application No. 60/385,972, filed on Jun. 5, 2002.

(51) Int. Cl. G01N 33/20

(52) U.S. Cl. 436/79

(57) ABSTRACT

The invention concerns arrays, systems and methods for screening compounds, compositions or conditions useful to treat or prevent kidney diseases. These methods are useful to discover, optimize and select compounds, compositions, or conditions that prevent, inhibit, induce, modify, or reverse physical-state transitions, particularly in-vivo physical-state transitions relating to disease causing processes such as development and growth of renal calculi. Such compounds, compositions, or conditions can be exploited to treat, prevent or manage the disease itself, the cause of the disease, or the symptoms of the disease.

### 1. FIELD OF THE INVENTION

The invention is directed to methods and systems for high throughput sample screening of conditions and compounds or compositions useful for preventing or treating urological diseases.

### 2. BACKGROUND OF THE INVENTION

2.1. Physical-State Transitions

Many serious diseases, conditions, or disorders of both humans and animals are associated with or by abnormal physical-state transition of benign, beneficial, or toxic substances, normally present in the body, such that various deposits or calculi result. These physical-state-transition disorders are a heterogeneous group of diseases. They include disorders caused by undesirable crystallization, bio-mineralization, polymerization, or calculus build up, such as bladder stones, kidney stones, gallstones, tartar build up on teeth, protein precipitation in body fluids, passage of aggregates based on their form or habit, and defects in bone formation or loss of bone mass.

Physical-state-transition diseases or conditions may also occur as a result of pathological degradations of substances in the body, for example, loss of bone mass caused by bone resorption or osteoporosis. Another instance of unfavorable biocrystallization or bioprecipitation concerns physiologically low-solubility pharmaceuticals or pharmaceuticals that complex with tissue or other bodily substances.

The occurrence of these undesirable physical-state transitions can be influenced by a number of factors, including environmental and dietary factors. For example, the deposition of calcium phosphate in solid form can be inhibited by the presence of pyrophosphate, even if the local concentrations of calcium and phosphate ions in a patient would otherwise promote such deposition. Similarly, formation of solids in the urinary system can be affected by altering the pH of the urine.

In general, the process of crystallization is one of ordering. During this process, randomly organized molecules in a solution, an amorphous substance, a melt, or the gas phase take up specific, ordered positions in a crystal lattice. The term precipitation is usually reserved for formation of amorphous substances that have no symmetry or ordering and cannot be defined by habits or as polymorphs. Bio-precipitation processes can result in organic deposits, such as plaques, fats, and other undesirable amorphous-substance buildup in the body. Both crystallization and precipitation result from the inability of a solution (e.g., body fluid) to maintain the substance in solution and can be induced by changing the state of the system in some way. Common parameters that can promote or discourage precipitation or crystallization include: pH; temperature; concentration; and the presence or absence of inhibitors or impurities.

Important events or processes in crystallization and precipitation are nucleation, growth, interfacial phenomena, agglomeration, breakage, and kinetics thereof. Nucleation results when the phase-transition energy barrier is overcome, thereby allowing a particle to form from a supersaturated solution. Growth is the enlargement of particles caused by, for example, incorporation or deposition of small molecules, ions, peptides, small proteins, and solids in or on an existing surface. The relative rate of nucleation and growth determine size distribution. Agglomeration is the formation of larger particles through two or more particles (e.g., crystals) sticking together. The thermodynamic driving force for both nucleation and growth is supersaturation, which is defined as the deviation from thermodynamic equilibrium.

At any point during crystallization, dissolution, stagnation, or precipitation processes, an adduct molecule can be incorporated into the matrix, adsorbed on the surface, or trapped within the particle or crystal. Such compositions are referred to as inclusions, for example, when crystals grow over another compound or material. Moreover, salts or polymorphs may also include inclusions. Whether a crystal forms in the body as an inclusion can have a profound effect on the clinical aspects of a disease, such as ease of removal from the body. For example, inclusions may dissolve more or less readily in bodily fluids or have different mechanical properties or strength than the corresponding non-inclusion containing substances.

Furthermore, the same compound can crystallize in different external shapes depending on various factors, such as the composition of the crystallization medium. These crystal-face shapes are described as the crystal habit, Crystal habit is an important factor as it has a large influence on the crystal's surface-to-volume ratio. As a result, crystals of the same compound exhibiting different habits may have the same internal structure and thus may have identical single crystal- and powder-diffraction patterns, but they can still exhibit different pathological (or pharmaceutical) properties. See, e.g., Haleblian, J. Pharm. Sci., 64:1269 (1975). For example, with calcium oxalate calculi the crystal size and shape, i.e., habit, can have a great effect on the clinical aspects of a disease, such as the ability (or inability) to urinate, and whether or how much pain the patient experiences, chronically and acutely (e.g., while attempting to urinate). Thus, discovering conditions or pharmaceuticals that affect crystal habit are needed.

Additionally, the same compound can crystallize as more than one distinct crystalline species (i.e., having a different internal structure and physical properties) or shift from one crystalline species to another. This phenomenon is known as polymorphism or pseudomorphism with distinct species known as polymorphs. Polymorphism as used herein includes true polymorphism and pseudomorphism. Polymorphs, salts, hydrates and solvates can exhibit different optical properties, different melting points, different solubilities, different chemical reactivities, different dissolution rates, and different bioavailabilities. Factors that affect polymorphism of foreign substances in the body are of clinical importance. For example, one polymorphic form may be more readily removed from the body—e.g., more soluble in body fluids—than another. Thus, conditions, compounds, or compositions that prevent shift to an unfavorable polymorph form or promote shift to a more favorable polymorph form are desirable.

Particles can form from solution in different sizes and size distributions depending on a number of conditions, including concentration, the presence of inhibitors or impurities, and other conditions. In general, smaller particles are more easily eliminated from the body. Thus, a need exists to discover compounds or compositions that promote small crystal size, which can be of clinical importance in treating or preventing diseases caused by solid deposits in the body.

2.2. Physical-State Transition Diseases or Disorders

Diseases, conditions, or disorders that can be characterized as involving physical-state-transitions can be quite diverse and may exhibit a complex and interconnected nature. A partial list of diseases, conditions, or disorders caused by physical-state transitions that result in deposition of disease-causing substances, including calcium oxalate calculi or crystallization, in the body for which there is a need to find compounds, compositions, or conditions to treat such diseases is, for instance, found in Cecil Textbook of Medicine, Eds. Goldman L. & Bennett J. C., 21^{st} Edition (2000), W. B. Saunders Co., Philadelphia; and Primer on the Metabolic Bone Diseases and Disorders of Mineral Metabolism, Second Edition, Ed. Favas, M. J. (1993), Lippincott-Raven Publisher, Philadelphia-New York, both incorporated by reference herein in their entirety.

Some crystals that are associated with physical-state transition or crystal deposition diseases, conditions, or disorders, as well as some of their diagnostic features, include: calcium pyrophosphate, which is typically 2-20 μm rod or rhomboid shaped crystals that are weak positive for crystal birefringence/elongation; apatite, which is typically chunks or globules about 2-25 μm in size that are non-birefringent; oxalate, which is typically 2-15 μm rod or bipyramidal shaped crystals that are positive for crystal birefringence/elongation; monosodium urate, which is typically 2-20 μm rod or needle shaped crystals that are bright negative for crystal birefringence/elongation; liquid-crystalline lipids, which are typically 2-12 μm maltese crosses that are positive for crystal birefringence/elongation; cholesterol, which has a notched rectangle shape, typically 10-80 μm in size, that can be either positive or negative for crystal birefringence/elongation; depot corticosteroids, which are typically 4-15 μm rod or irregularly shaped crystals that can be bright positive or negative for crystal birefringence/elongation; immunoglobulins and other proteins, which are typically 3-60 μm rod or irregularly shaped crystals that can be positive or negative for crystal birefringence/elongation; and Charcot-Leydon crystal protein (galectin 10), which is typically 10-25 μm spindle shaped crystals that can be positive or negative for crystal birefringence/elongation. See, Cecil Textbook of Medicine, Eds. Goldman L. & Bennett J. C., 21^{st} Edition (2000), W. B. Saunders Co., Philadelphia.

The composition of disease- or disorder-causing stones or calculi that form in a patient's tissues or organs, such as the kidney or ureters, is variable. In general, about three-fourths of all stones are composed of calcium oxalate: 35% are pure calcium oxalate (calcium oxalate monohydrate or calcium oxalate dihydrate or both); 40% are calcium oxalate with hydroxyapatite or carbonate apatite; and 1% are calcium oxalate with uric acid. Four percent of all calculi stones are apatite or hydroxyapatite [Ca₁₀(PO₄)₆(OH)₂] and 1% are brushite [CaHPO₄ 2H₂O]. The non-calcium-containing crystal stones are struvite (magnesium ammonium phosphate) and comprise 8% of all stones. In addition, eight percent of all stones are composed of uric acid and 2% of cystine. In rare cases stones may be composed of acid ammonium urate or xanthine or insoluble drugs.

One of the most common and prototypic diseases or conditions caused by physical-state transition (or crystal deposition) is gout, which involves calculi or stones that typically consist of uric acid and its salts. For example, gout can be caused by monosodium urate crystals depositing in joints and other connective tissue causing gout. Monosodium urate crystals are rods or needles up to 15 to 20 micrometers in length and exhibit bright birefringence with negative elongation when viewed with compensated polarized light.

Gout commonly involves a variety of lower extremity joints. Chronic or recurrent acute gout can be polyarticular and can mimic rheumatoid arthritis. Crystals are often present in joint fluid even between attacks and may contribute to low-grade inflammation and joint damage. Moreover, the presence of uric acid crystals in the kidneys can cause renal failure.

Current treatment options for gout are somewhat limited with a common treatment comprising administration of colchicine for acute gout notwithstanding its side effects of nausea, vomiting and diarrhea. A more modern treatment is the use of non-steroidal anti-inflammatory drugs, and in particular the agent indomethacin. In addition, xanthine oxidase inhibitors, such as allopurinol, are administered to control high levels of uric acid.

A number of disease, conditions, or disorders are associated with oxalate crystal deposition or oxalate calculi, including without limitation hyperoxaluria, nephrolithiasis (kidney stones or calculi), and ureterolithiasis (kidney stones found in the ureter). Oxalate crystals (e.g., calcium oxalate) are most commonly found in kidneys, liver, urinary tract, bone, skin, vessels, and vessel walls, and less commonly in eyes, other organs, and inside the joints or synovial fluids (e.g., in association with acute or chronic arthritis). See, Maldonado et al., Curr. Rheumatol. Rep., 4(3):257-264 (2002).

Hyperoxaluria (and its accompanying oxalosis) is a disease or condition where patients suffering from the same have significantly elevated levels of oxalate in their blood and urine. There are a number of forms of hyperoxaluria, including primary hyperoxaluria Type I and Type II, a poorly defined hyperoxaluria Type III, acquired hyperoxaluria, and absorptive or enteric Hyperoxaluria. A common feature among all forms of hyperoxaluria is an excessive excretion of oxalate in the urine, which typically is accompanied by oxalosis, which is the excessive accumulation of oxalate in the body.

Primary hyperoxaluria Type I (also known as PHI) is an autosomal recessive inherited disease and is a rare metabolic disease caused by the liver overproducing oxalic acid, which is then excreted in the urine of the affected patient. Once inside the kidneys, the excess oxalic acid combines with calcium, causing calcium oxalate stones or calculi in the urinary tract. These stones or calculi can cause patients both acute and chronic pain in the kidneys, bladder, and in the ureters as the patient attempts to pass or eliminate the stones or calculi. In PHI, a liver enzyme that is necessary for the body to eliminate the precursors that lead to excessive oxalate production is absent. The missing enzyme is called alanine: glyoxalate transferase or “AGT”, and it is normally found in a special area of liver cells called the peroxisome. At present, it is not possible to synthesize the missing enzyme.

Long-term prognosis for PHI patients is bleak and it is often fatal unless the patient can obtain both liver transplants (in effort to get normal production of AGT) and kidney transplants to replace their kidneys, which are damaged due to the excessive precipitation of calcium oxalate and stone formation and growth.

Primary hyperoxaluria Type II (also called PHII) is also an autosomal recessive disorder caused by a defective gene in liver cells and other cells. PHII is a milder disease and does not usually lead to the long-term kidney impairment that is seen with PHI.

In patients suffering from hyperoxaluria, once a patient's kidneys cease to function, the disease progresses to oxalosis. Since the kidneys are no longer functioning to eliminate calcium oxalate from the patient's body, the calcium oxalate crystals or calculi are deposited in other areas of the body, including other major organs, the eyes, bones, and joints, which results in the patient suffering from any number of additional diseases and conditions, and eventually death if the patient does not receive liver and/or kidney transplants

Other physical-state transition diseases or disorders involving oxalate are nephrolithiasis and ureterolithiasis, which are fairly common and can result in extreme pain and discomfort to a patient. Kidney stones are a major cause of morbidity in the United States and elsewhere, and approximately 12% of the population of the United States will have a kidney stone at some time in their lives. The economic impact is more than $2 billion dollars per year. Kidney stones are two to three times more common in men than in women and in the U.S. are most common in the southeast.

While some kidney calculi pass out of the body without any intervention (sometimes referred to as “silent stones”), larger calculi can cause extreme and constant pain, block the flow of urine, cause urinary tract infection, damage kidney tissue or cause constant bleeding, and these calculi can continue to grow as long as they remain in the patient.

Kidney calculi comprise a hard mass of crystals (typically calcium oxalate) that separate from the urine and build up on the inner surfaces of the kidney. The urine of normal patients contains components that work to prevent these crystals from forming or keep the crystals from growing so that they are small enough to pass through the urinary tract and pass out of the body in the urine without notice.

Many different mechanisms can result in physical-state-transitions leading to formation of calcium oxalate calculi. Calcium oxalate calculi can also form by a physical-state shift from one solid to a more unfavorable solid, for example, a polymorphic shift. Addition of a promoter or absence of an inhibitor in biological fluids can result in the formation of calcium oxalate calculi in the form of precipitates and crystals, including composite materials.

In nephrolithiasis, calcium oxalate calculi typically form as crystals that may then nucleate, aggregate, agglomerate, adhere to cell surfaces, or otherwise grow without being excreted. In particular, formation of kidney stones or calculi can result from (a) initial formation of crystals (nucleation), (b) reduced effects of normal urinary constituents that inhibit crystal growth and aggregation; (c) the presence of substances promoting crystal growth and aggregation; and (d) the processes that mediate and regulate crystal attachment to the renal tubule or nephron.

There are ongoing efforts to improve treatment for renal stones. For instance, U.S. Pat. No. 5,776,348 issued on Jul. 7, 1998 discloses a system for analysis of mineral precipitation formation with a view to inhibit undesirable formations of struvite to treat kidney stones and/or calcium phosphate. Similarly, Chinese patent No. CN1241411 published on Jan. 19, 2000 discloses a medicine comprising Cortex, Phellodendri, talcum, Herba Violae, bobelia chinesis, Radix Polygalae, and eighteen other Chinese medicinal herbals to treat lithiasis. The U.S. Pat. No. 5,304,496 issued on Apr. 19, 1994 discloses an aspartic acid rich protein in human urine as a modulator of mineralization and of use in the treatment of kidney stone disease.

In a similar vein, Bulgaria Patent No. BG103400 published on Apr. 30, 2001 discloses growing stone-like compounds on a monocrystalline quartz substrate from supersaturated solutions set in oscillation by piezoelectric effect. Slovenia Patent No. SI9800012 published on Aug. 31, 1998 discloses an agent for treating kidney stones. The agent is prepared by dissolving potassium citrate in distilled water and pyridoxal chloride followed by mixing with mineral water having high magnesium content.

U.S. Pat. Nos. 5,618,917 and 6,043,216 issued to Toback et al. on Apr. 8, 1997 and Mar. 28, 2000, respectively, disclose an autocrine adhesion inhibitor, CA1, for blocking adhesion to the cell surface and administration in-vivo to prevent nephrolithiasis.

Some of the presently-used treatment options for different types of renal stones include: for all stones, increased fluid (e.g., water) intake; for calcium oxalate and brushite (CaOxICaHPO₄) stones, administration of citrate compounds, such as potassium citrate; for idiopathic hypercalciuria, treatments include i) controlled protein, sodium, and calcium diets, ii) thiazide diuretics and related drugs, iii) oral phosphate, and iv) sodium cellulose phosphate; for uric acid stones, administration of allopurinol or potassium citrate; for struvite stones, extracorporeal shock wave lithotripsy (ESWL), percutaneous nephrostolithotomy, or administration of acetohydroxamic acid; for cystine stones/cystinuria, administration of tiopronin or penicillamine; for hypercitraturia, including renal tubular acidosis and ileostomy or small bowel malabsorption, administration of potassium citrate; for dietary hyperoxaluria, reduced oxalate diet; for enteric hyperoxaluria, a low fat diet, calcium supplement, and cholestyramine; for primary hyperoxaluria, administration of pyridoxine; and for hyperuricosuria, administration of allopurinol or potassium citrate. The reliability of these treatments for various types of stones is generally quite variable and the expected results unknown from patient to patient. See, e.g., Cecil Textbook of Medicine, Eds. Goldman L. & Bennett J. C., 21^{st} Edition (2000), W. B. Saunders Co., Philadelphia. As a result, at present, the treatment for many types of renal calculi is imperfect and the occurrence of renal calculi continues to cause significant morbidity and high cost to the health care system associated with emergency room visits, etc. Thus, there is a need for more effective treatment and, especially, methods of prevention of the formation of renal calculi.

There remains a need for both a better understanding of the processes involved in nephrolithiasis and other diseases, disorders, and conditions associated with pathological physical-state transitions of calcium oxalate, as well as a method to treat or prevent such diseases. Thus, there is a need for cost- and time-effective methods to discover conditions, compounds, or compositions that prevent, inhibit, or modify nucleation, formation, and/or growth of pathogenic calculi underlying such diseases, particularly calcium oxalate calculi.

The present invention addresses these and other concerns.

### 3. SUMMARY OF THE INVENTION

In one embodiment, the invention encompasses practical and cost-effective systems or methods to rapidly produce and/or screen hundreds, thousands, to hundreds of thousands of samples per day, wherein the samples comprise experiments designed to identify inhibitors or modifiers of crystallization, agglomeration, adhesion, or migration processes involved in kidney disease or disorders. Such vast numbers of samples are produced and screened by high throughput techniques disclosed in this and related applications. These methods, in contrast to the accidental and sporadic discovery of isolated treatments in the past, provide an extremely powerful tool for the rapid and systematic analysis, optimization, selection, or discovery of conditions, compounds, or compositions that prevent, inhibit, induce, modify, or reverse physical-state transitions, preferably, physical-state transitions that cause diseases, conditions, or disorders in humans and animals, and more preferably physical-state transitions relating to kidney calculi (e.g., kidney stones), calcium oxalate calculi or crystals, or related diseases or disorders, such as kidney stones, nephrolithiasis, ureterolithiasis, hyperoxaluria, or oxalosis.

In a further embodiment, the invention encompasses discovery, selection, or optimization of compounds or compositions that prevent, inhibit, or modify nucleation, crystallization, precipitation, formation, or deposition of inorganic and/or organic substances, or modification of inorganic and/or organic solids, particularly calculi involved in diseases, disorders or conditions in humans or animals, including without limitation nephrolithiasis, ureterolithiasis, hyperoxaluria, or oxalosis, particularly calcium related calculi, such as calcium oxalate calculi. The invention further encompasses the use of such compounds or compositions to treat (e.g., reverse), manage, or prevent disease itself, recurrence of disease, the cause of disease, or the symptoms of such diseases, including diseases such as kidney stones, nephrolithiasis, ureterolithiasis, hyperoxaluria, and oxalosis.

The invention further encompasses a method for the discovery of physiological conditions (e.g., pH, salt concentration, protein concentration, hormone concentration, etc.) that inhibit, prevent, or modify crystallization, precipitation, nucleation, formation, or deposition of inorganic and/or organic substances, particularly calculi (such as kidney calculi or calcium oxalate calculi) that cause diseases, conditions, or disorders in humans and animals, such as nephropathy, nephrolithiasis, ureterolithiasis, hyperoxaluria, or oxalosis. Once such physiological conditions are identified, the invention further contemplates the use of drugs or other therapies to achieve these conditions, and thereby prevent, manage, or treat the disease, condition, or disorder itself, recurrence thereof, its cause, slow or modify progression, or the symptoms of the disease, condition, or disorder.

The invention also encompasses methods to discover compounds, compositions, or physiological conditions that prevent, inhibit, or modify a change in physical state of a solid substance, for example, prevention, inhibition, or modification of nucleation, crystallization, crystal growth, agglomeration, mineralization, adhesion, or migration of the solid substance, particularly, calcium oxalate, in the bladder, kidney, or the urinary or biliary passages or tracts. Modification of crystallization of the invention encompasses modification of habit as well as polymorphism.

The invention further encompasses methods to discover compounds, compositions, or physiological conditions that prevent, inhibit, or modify unfavorable biocrystallization or bioprecipitation of substances in the kidney or renal system.

In an embodiment, the invention encompasses a method to discover conditions, compounds, or compositions that prevent, inhibit, or modify nucleation, crystallization, precipitation, formation, crystal growth, or deposition of crystals that are components of calculi, comprising:
(a) preparing an array comprising at least 24 samples, preferably 384 samples, each sample comprising a medium, with or without “seed” crystals of calcium oxalate, and one or more components to induce crystals that are components of a calculus;
(b) processing one or more of the samples to induce nucleation, crystallization, precipitation, formation, growth, agglomeration, or deposition of the crystals;
(c) screening the array by analyzing the processed samples to detect growth of the seed crystals, the absence of nucleation, crystallization, precipitation, formation, crystal growth, agglomeration, or deposition of crystals, or both; and selecting the samples wherein the seed crystals did not grow, or nucleation, crystallization, precipitation, formation, crystal growth, agglomeration, modification or deposition of crystals did not occur to identify the conditions, compounds, or compositions. In one embodiment, the foregoing method can be performed iteratively to discover the conditions, compounds, or compositions that are most useful or advantageous for a particular purpose

In a preferred embodiment, specific high throughput methods identify compounds, particularly small molecules, that modify or prevent one or more of the following processes involved in the formation of calculi, such as calcium oxalate calculi: (a) crystal growth; (b) crystal nucleation; (c) crystal agglomeration; (d) adhesion to tubular structures and cell surfaces; and (e) migration of crystals. Preferably, the compounds modify or prevent at least crystal growth.

The invention further encompasses the use of compounds identified by the screening methods or systems in the treatment, management, and/or prevention of diseases, conditions, or disorders caused by calculi or physical-state transitions, including nephrolithiasis, ureterolithiasis, hyperoxaluria, or oxalosis. In one embodiment, the invention encompasses several small organic molecules having a molecular weight less than 750 daltons preferably less than 500 daltons and their use in novel methods of treating or preventing nephrolithiasis, and disease, conditions, or disorders associated with or symptoms associated with nephrolithiasis. The invention also encompasses pharmaceutical compositions comprising one or more of these molecules and a pharmaceutically acceptable excipient, carrier, or diluent. These compositions can be adapted for any suitable route of administration, and preferably for oral or parenteral administration.

These and other features, aspects, and advantages of the invention will be better understood with reference to the following detailed description, examples, and appended claims.

### 4. DESCRIPTION OF THE FIGURES

FIG. 1 is an illustrative flow chart describing a method for preparing supersaturated solutions for high throughput screening and analyzing the samples.

FIG. 2 is an illustrative flow chart for high throughput screening of supersaturated solutions having putative inhibitors or promoters of crystal nucleation and/or growth.

FIG. 3 is a turbidity based measurement of crystal nucleation and growth in supersaturated calcium oxalate solutions prepared in accordance with the method of FIG. 1.

FIG. 4 is an illustrative flow chart describing a method for high throughput screening for adhesion (or lack thereof) of crystals comprising calcium oxalate to kidney epithelium or related cells.

FIGS. 5A and 5B are illustrative RAMAN spectra corresponding to several crystal compositions based on components of normally occurring kidney stones, which illustrates the utility of Raman in the methods of the invention.

FIGS. 6A and 6B are illustrative birefringence photographs showing detection of crystal formation and/or growth in supersaturated calcium oxalate solutions, which further illustrates the utility of this detection method within the invention.

FIG. 7 is an illustrative flow chart describing a method for high throughput screening of supersaturated calcium oxalate solutions to detect reversible and irreversible inhibitors of crystal growth.

FIG. 8 is an illustrative flow chart describing a method for high throughput screening of supersaturated calcium oxalate solutions to detect reversible and irreversible inhibitors or promoters of crystal nucleation.

FIGS. 9A and 9B present a listing of various compounds evaluated in accordance with the described methods and systems that modify the nucleation, growth, habit, or other properties of crystals in supersaturated calcium oxalate solutions.

FIG. 10 illustrates the effect of citrate on growth and nucleation of crystals in supersaturated calcium oxalate solutions as measured by turbidity at 620 nm.

FIG. 11 is an example of turbidity based assay showing crystal nucleation and growth in supersaturated calcium oxalate solutions prepared in accordance with the method of FIG. 1.

### 5. DETAILED DESCRIPTION OF THE INVENTION

As an alternate approach to traditional methods for exploring pharmaceuticals as potential therapeutics for diseases, conditions, or disorders involving calculi (such as calcium oxalate calculi), including nephrolithiasis, ureterolithiasis, hyperoxaluria, and oxalosis, applicants have developed practical and cost-effective methods to rapidly produce and screen hundreds, thousands, to hundreds of thousands of samples per day, wherein the samples are specifically designed to identify inhibitors or modifiers of the processes involved in such diseases. These methods are useful to improve, select, and discover compounds, compositions, or conditions that prevent, reverse, or modify calculi (e.g., renal calculi). For example, these methods are useful to identify, optimize, select, and discover compounds, compositions, or conditions that prevent, inhibit, or modify nucleation, crystallization, precipitation, crystal growth, formation, modification, or deposition of inorganic and/or organic substances, particularly calcium oxalate. Such conditions, compounds, or compositions can be exploited to treat (e.g., reverse), manage, or prevent the disease itself, recurrences of the disease, the cause of the disease, or the symptoms of the disease.

In the preferred embodiment, samples are prepared in a grid or array (i.e., an ordered set of components) such as a 24, 36, 48, 96, 384, or 1536 well plate. Each sample in the array comprises a medium and at least one of the samples comprises a substance capable of forming crystals that are components of calculi of the ureter, bladder, kidney, or renal system.

The array or selected samples therein can be subjected to processing parameters. Examples of processing parameters that can be varied include temperature, temperature gradient, time, the identity or the amount of the crystal-forming substance, the presence or absence of “seed crystals” comprised of the same or different components as are in the solutions, the identity or the amount of the medium, or the identity or the amount of the components, i.e., concentrations of the components or solutions.

Before, during, and/or after processing, each sample in the array or processed array can be screened to assess the components therein to determine the presence or absence of a solid or crystal, and to determine whether a change in physical state occurred, particularly a change in the physical state of calcium oxalate crystals or calculi. The presence or absence of a solid or crystal can be assessed by turbidity, using a device such as a spectrophotometer. Simple visual analysis can also be conducted such that the types of analysis include but are not limited to photographic analysis, turbidity evaluation, particle sizing, X-ray diffraction, birefringence evaluation, Raleigh scattering evaluation, spectroscopic evaluation, and the like. The nature of the solid forms or crystals can be determined including size, habit, number of particles, composition and the like. Crystal forms, different polymorphs, and other amorphous solids are then determined. The samples containing a solid can then be screened to analyze the solid's properties, such as structural, physical, biological, or chemical properties. In a preferred embodiment, the samples are screened to define the conditions, compounds, or compositions, that prevent, inhibit, or modify nucleation, crystallization, precipitation, crystal growth, formation, or deposition of inorganic and/or organic substances, or modification of inorganic and/or organic solids, particularly renal calculi or components (such as calcium oxalate crystals) thereof. However, the methods may also be adapted for screening related to other calculi. Systems employing these methods have been designed to rapidly, systematically, and inexpensively screen such samples. The methods and systems are widely applicable.

In one embodiment, the methods encompass the screening of samples comprising natural, synthetic, or semi-synthetic urine. The urine may be of animal or human origin. The urine can be used to screen known or novel small organic compounds, peptides, proteins, nucleotides, polynucleotides, saccharides, oligosaccharides, and other molecules for their ability to cause, reduce, inhibit, or modify nucleation, crystallization, precipitation, crystal growth, formation, or agglomeration of calcium oxalate crystals or calculi.

Urine is a highly diverse composition that is subject to variations from patient to patient, as well as variations at different times in the same patient, and such variations can not only result from disease or other conditions, but also from numerous other factors, including without limitation, diet, water intake and/or excretion, sleep, temperature, and the like.

Thus, another embodiment of the invention encompasses a method for creating a lithogenicity index for natural, synthetic, or semi-synthetic urine, comprising collecting samples of urine from normal and diseased patients, screening the samples for one or more components, and concentrations thereof, that demonstrate enhanced lithogenicity, and compiling the results of the screening to create a lithogenicity index for each of the components, and concentrations thereof.

Another embodiment of the invention encompasses a method for determining lithogenicity of a natural, synthetic, or semi-synthetic urine sample, comprising screening the sample, identifying the component or components of the sample and the concentration of each component, and using the results to create a lithogenicity profile for the sample, and comparing the lithogenicity profile to a lithogenecity index to determine whether the sample demonstrates enhanced lithogenicity. The invention further encompasses methods for reducing the lithogenicity of urine in a patient comprising administering to said patient an amount of one or more compounds, or pharmaceutical compositions comprising the same, that eliminate or alter the concentration of a component or components in urine that demonstrates enhanced lithogenicity.

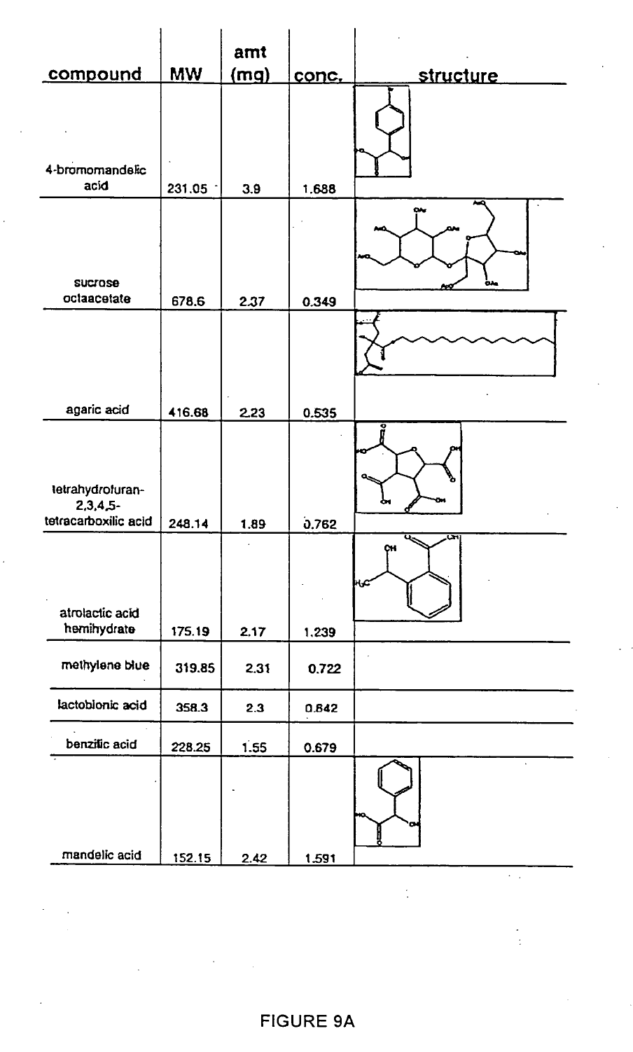

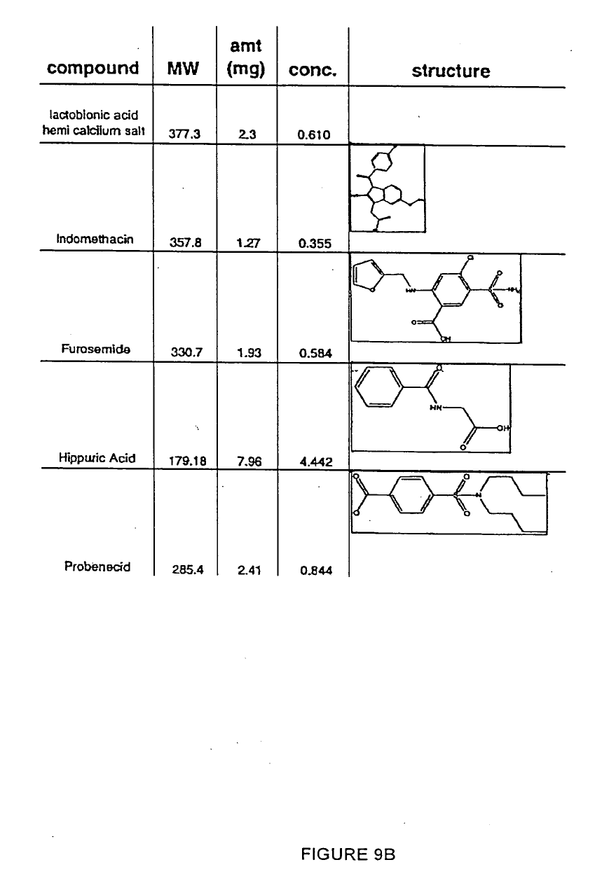

In another embodiment the invention encompasses the use of the small organic molecules of FIGS. 9A and 9B to inhibit or modify calcium oxalate crystallization or precipitation or agglomeration in urine. The invention further encompasses the use of these compounds or pharmaceutical compositions thereof in the treatment or prevention of nephrolithiasis or disease associated therewith comprising the administration of a therapeutically effective amount of one or more of said compounds to a human in need thereof. A therapeutically effective amount is an amount sufficient to ameliorate one or more symptoms of the nephrolithiasis or to enable the passing of any calcium oxalate particle that was not previously passing out of the renal system. This amount can be calculated based upon the patient's size, weight, and overall condition. The physician or clinician will know when to reduce or otherwise modify the dose or dose frequency dependent upon patient response.

5.1. Helpful Definitions

As used herein, the term “array” means a plurality of samples, preferably, at least 24 samples. Each sample comprises a medium, and at least one of the samples comprises a substance that is crystalline or capable of crystallizing, such as calcium oxalate crystal(s)/precipitate. Preferably, each sample comprises calcium oxalate, with the exception of negative controls. Each sample can have different components or concentrations of components. The samples are associated under a common experiment. An array can comprise 24, 36, 48, 96, 384, or more samples, preferably 1000 or more samples, more preferably, 10,000 or more samples. An array is typically comprised of one or more groups of samples also known as sub-arrays. For example, a sub-array can be a 96-tube plate of sample tubes or a 96-well plate of sample wells in an array comprising 100 plates.

Arrays can be assembled by preparing a plurality of samples using standard addition and mixing techniques. If desired, each sample, selected samples, or selected sub-arrays can be subjected to the same or different processing parameters. For example, an array can be processed to prevent, inhibit, accelerate, slow, or modify nucleation, crystallization, precipitation, crystal growth, formation, or deposition of the calcium oxalate crystals or calculi, or to promote modification of the calcium oxalate crystals or calculi, or to promote or cause the nucleation or formation of calcium oxalate crystals or calculi that are flow-compatible, i.e., capable of being passed by a patient in the normal fashion. Arrays can be processed by a variety of methods readily ascertainable by one skilled in the art according to the objective of the experiment. For example, the array can be stored at a particular temperature, such as room temperature. The samples can be subjected to a temperature gradient, such as cooling the sample. Or the pH can be adjusted by adding acidic or basic components. The array can also be subjected to standard methods well known in the art to prevent, inhibit, or modify crystallization, precipitation, formation, or deposition of the calcium oxalate calculi, or modification of the calcium oxalate calculi, for example, but not limited to, ultrasound, shock-waves, or laser energy.

In one embodiment, one or more tubes or wells in an array are modified or etched to contain a select surface or a surface nucleation site that can be used to control or direct nucleation of crystals, such as calcium oxalate crystals. These tubes or wells in an array would be predisposed toward nucleation of crystals, and thus compounds, compositions, or conditions that mask the select surface or nucleation site, thereby preventing or inhibiting crystal nucleation in such tubes or wells can be identified as compounds useful in treating, managing, or preventing diseases, conditions, or disorders involving crystal deposition or calculi (such as calcium oxalate calculi), including nephrolithiasis, ureterolithiasis, hyperoxaluria, and oxalosis.

As used herein, the term “disease-causing substance” means any solid, semisolid, paste, gel, plaque, macrophage, macromolecule, protein, peptide, metabolite, polynucleotide, or liquid in dissolved or undissolved form, that can crystallize, precipitate, or otherwise accumulate or deposit crystals or calculi, in particular, calcium oxalate crystals or calculi, in a human or animal, thereby causing or aggravating a disease process. Examples of disease-causing substances include, but are not limited to, calcium salts and compositions thereof, such as calcium phosphate, calcium carbonate, calcium pyrophosphate, calcium oxalate, and kidney stones; magnesium salts and compositions thereof, such as magnesium ammonium phosphate; uric acid and salts thereof or hydrates and mixtures thereof.

Calcium oxalate crystals and calculi can form in vivo—and thus can be isolated from an animal, plant, tissue, or cell culture—or they can be prepared in a laboratory setting to mimic one or more physical, chemical, or structural properties of those formed in vivo. Calcium oxalate calculi derived from animals and plants are often complex mixtures, thus, those prepared in a laboratory setting will usually only approximate those formed in vivo.

In the context of kidney diseases, conditions, and disorders, disease-causing substances include calculi, including those containing calcium oxalate, such as in the bladder, kidney, or passage or the urinary or biliary tracts. Kidney diseases and disorders include, but are not limited to, kidney stones, nephrolithiasis, ureterolithiasis, and other diseases, conditions, or disorders involving calcium oxalate deposition or calculi, such as hyperoxaluria and oxalosis.

As used herein the term “medium” is the solution in which the inhibition or promotion of various phase transitions or changes of physical state are tested. Preferably, a medium will be chosen to mimic the physiologic conditions under which such changes occur in vivo. Thus, the precise composition of the medium will depend on which phase transition or change in physical state is being investigated. Thus, for example the formation of renal calculi will be investigated in solutions that are similar to the composition of urine in various parts of the kidney and bladder.

The exact composition of the medium employed depends on the nature of the phase transition or change in physical state being investigated, but will be primarily water based. Medium includes supersaturated calcium oxalate containing media for nucleating and/or growing crystals. Medium may vary in pH, electrolyte concentration, protein concentration, and the presence or absence of various organic and inorganic compounds such as cholesterol, sodium ions, potassium ions, calcium ions or mucins, polysaccharides or proteins. These media may be obtained from natural sources such as blood plasma, urine, and joint space fluid or surrogate media reflecting one or more properties of interest can be prepared by means well known to one of ordinary skill in the art.

Thus, in the case of kidney diseases, conditions and disorders, the medium preferably contains urine, which may, for instance, be artificial or human. Preferably, the medium contains calcium oxalate, including supersaturating levels of calcium oxalate. In addition, in the context of kidney diseases and disorders, medium may also be suitable for culturing cells, for instance as monolayers, suspensions, or as part of a tissue or organ, including a perfused organ.

As used herein, the term “sample” means at least a medium isolated at a particular location or site, preferably, further comprising calcium oxalate or a test component or both. A sample can comprise multiple disease-causing substances. In addition, a sample can comprise one or more components. Preferably, the amount of the disease-causing substance, such as calcium oxalate, is less than about 100 milligrams, more preferably, less than about 1 milligram, even more preferably, less than about 100 micrograms or even 10 micrograms. Preferably, the sample has a total volume of about 5 μl to about 500 μl, more preferably, about 10 μl to about 200 μl.

A sample can be contained in any container or holder, or present on any material or surface, or absorbed or adsorbed in any material or surface. The only requirement is that the samples are isolated from one another, that is, located at separate sites. In one embodiment, samples are contained in sample wells in standard sample plates, for instance, in 24, 36, 48, 72, 96, 384, or 1536 well plates (or filter plates) commercially available, for example, from Millipore, Bedford, Mass.

According to one embodiment of the invention, the amounts or the identity of the medium, the components, or calcium oxalate can vary between samples. For example, within an individual array or sub-array, one or more of the samples can differ from one or more of the other samples with respect to: (i) the presence or the amount of calcium oxalate; (ii) the identity or the amount of the medium; or (iii) the identity or the amount of at least one of the components.

Such amounts and identities will differ between samples when they are intentionally varied to induce a measurable change in the sample's properties. Thus, according to the invention, minor variations between samples, such as those introduced by slight weighing and measuring errors, are not considered intentionally varied.

Thus, in the case of kidney diseases, conditions, and disorders, the sample preferably contains urine, which may, for instance, be artificial, non-human, or human, along with various levels of the relevant disease-causing substance and calcium oxalate. Preferably, the level of calcium oxalate includes supersaturating levels suitable to result in crystallization of calcium oxalate crystals or evaluation of inhibition thereof.

As used herein, the term “component” means any substance that is combined, mixed, or processed in the medium comprising a sample. A single component can exist in one or more physical states. Examples of suitable components include, but are not limited to, compounds and compositions that prevent, inhibit, or modify precipitation, formation, crystallization, crystal growth, or nucleation of inorganic and/or organic substances; compositions and compounds that promote modification of inorganic and/or organic solids; nucleation promoters (also known as crystallizing aids), such as seed crystals or surfactants, and combinations thereof; compositions or compounds that affect crystal habit; nutrients, such as vitamins and minerals; small molecules (i.e., molecules having a molecular weight of less than about 1000 g/mol), such as pharmaceuticals (e.g., ursodeoxycholic acid; diuretic agents, thiazides, and allopurinol); large molecules (i.e., molecules having a molecular weight of greater than about 1000 g/mol), such as oligonucleotides, proteins, and peptides; aminoglycans; hormones; steroids; matrix and connective tissue, such as cartilage and collagen; biological-membrane extracts; chelating agents, such as EDTA; anti-dental-calculus agents; excipients; solvents, including without limitation organic solvents, aqueous solvents or mixtures thereof; water; salts; acids; bases; gases; and stabilizers, such as antioxidants.

The term “component” also encompasses disease-causing solids, which, as discussed herein, can be added to samples according to certain embodiments of the invention. The term “component” further encompasses the ingredient or one of the ingredients in the sample medium—in dissolved or undissolved form—that can induce or result in nucleation, crystallization, precipitation, crystal growth, formation, or deposition of a crystals or calculi within the sample. Preferably, the crystals or calculi comprise calcium oxalate, such as calcium oxalate crystals or calculi that can be found in the bladder, kidney, or in passage through the urinary or biliary tracts of humans or animals. Moreover, urine is known to naturally contain nucleation and growth inhibitors of crystals or calculi of compounds such as calcium oxalate, and thus compounds or substances that promote in vivo nucleation or crystal growth in patients may be interfering with or inhibiting the naturally-occurring inhibitors. Thus, compounds or substances that are inhibitors of naturally-occurring nucleation or crystal growth inhibitors and the like are also intended to be included in components directed to modulating the inhibition mechanism for preventing the deposition of calcium oxalate calculi. Thus, the term “component” encompasses fractions prepared from urine. The urine may be from humans or non-humans. The urine may also be from one or more patients susceptible to formation of kidney stones or calcium oxalate deposits, or from normal subjects.

As used herein, the term “processing parameters” means the physical or chemical conditions to which a sample is subjected and the time during which the sample is subjected to such conditions. Mechanical preparation of nucleation material by crushing, sonication, cavitation, powdering, and the like of synthetic or natural stones/crystals is included within processing parameters. The period of incubation is also a processing parameter and means the time that a sample is given to undergo a change in physical state. For example, an array of samples can be incubated for 3 days at normal human-body temperature, then analyzed for a change in physical state. Processing parameters include, but are not limited to, adjustments in time of incubation, temperature, pressure, pH, chemical environment, concentration, subjecting the samples to a nucleation event, ultrasound, shock waves, laser energy, or mechanical stimulation, or any other conditions that can induce a change in physical state. Processing also includes adjusting the concentration of components, adding various additional components, or adjusting the composition or amount of the medium during incubation. Processing also includes parameters such as adjusting the oxygen tension or oxygen vapor pressure. A sample can be processed to prevent, inhibit, or modify nucleation, crystallization, precipitation, crystal growth, formation, or deposition of inorganic and/or organic substances, or to promote modification of existing inorganic and/or organic solids, particularly calcium oxalate crystals or calculi, such as renal calculi particularly those containing calcium oxalate.

Sub-arrays or even individual samples within an array can be subjected to processing parameters that are different from the processing parameters to which other sub-arrays or samples, within the same array, are subjected. Processing parameters will differ between sub-arrays or samples when they are intentionally varied to induce a measurable change in the sample's properties. Thus, according to the invention, minor variations, such as those introduced by slight adjustment errors, are not considered intentionally varied.

As used herein, the term “property” means a structural, physical, pharmacological, or chemical characteristic of a sample, preferably, a structural, physical, pharmacological, or chemical characteristic of a calcium oxalate crystal or calculus in a sample. Structural properties include, but are not limited to, whether a calcium oxalate crystal or calculus is crystalline or amorphous, and if crystalline, the polymorphic form and a description of the crystal habit. Structural properties also include the composition, such as whether a calcium oxalate crystal or calculus comprises a hydrate, solvate, or a salt, the degree of mineralization, and identity of the minerals.

Another important structural property is the surface-to-volume ratio and the degree of agglomeration of the particles. Surface-to-volume ratio decreases with the degree of agglomeration. It is well known that a high surface-to-volume ratio improves the solubility rate and ease of bodily elimination of calcium oxalate calculi and calcium oxalate. Small-size particles have high surface-to-volume ratio. The surface-to-volume ratio is also influenced by the crystal habit, for example, the surface-to-volume ratio increases from spherical shape to needle shape to dendritic shape. Porosity also affects the surface-to-volume ratio, for example, calcium oxalate calculi having channels or pores (e.g., inclusions, such as hydrates and solvates) have a high surface-to-volume ratio.

Still another structural property is particle size and particle-size distribution. For example, depending on concentrations, the presence of inhibitors or impurities, and other conditions, particles can form from solution in different sizes and size distributions. Particulate matter, produced by precipitation or crystallization, has a distribution of sizes that varies in a definite way throughout the size range. Particle- and crystal-size distribution is generally expressed as a population distribution relating to the number of particles at each size. In calcium oxalate calculi, particle and crystal size distribution have very important clinical aspects. For example, smaller particles are more easily eliminated from the body and have higher surface-to-volume ratio that allows easier dissolution in bodily fluids. Thus, compounds or compositions that promote small crystal size can be of clinical importance in treating or preventing diseases caused by calcium oxalate calculi in the body.

Pharmacological or biological properties include, but are not limited to, toxicity metabolic profile, excretion, accumulation, adhesion to cells, tubular structures and migration thereon or relative thereto.

Physical properties include, but are not limited to, melting point, solubility, strength, hardness, compressibility, compactability, and resistance to energy forms, such as ultrasound, shock waves, and laser energy. Physical stability refers to a compound's or composition's ability to maintain its physical form, for example maintaining particle size; maintaining crystal or amorphous form; maintaining complexed form, such as hydrates and solvates; resistance to absorption of ambient moisture; and maintaining of mechanical properties, such as compressibility and flow characteristics. Methods for measuring physical stability include spectroscopy, sieving or testing, microscopy, sedimentation, stream scanning, and light scattering. Polymorphic changes, for example, are usually detected by differential scanning calorimetry or quantitative infrared analysis. For a discussion of the theory and methods of measuring physical stability see Fiese et al., in The Theory and Practice of Industrial Pharmacy, 3rd ed., Lachman L.; Lieberman, H. A.; and Kanig, J. L. Eds., Lea and Febiger, Philadelphia, 1986 pp. 193-194 and Remington's Pharmaceutical Sciences, 18th Edition, ed. Alfonso Gennaro, Mack Publishing Co. Easton, Pa., 1995, pp. 1448-1451, both of which are incorporated herein by reference. Flow properties of particles through liquids is also a relevant physical property. For instance, flow properties of disease-causing solids in bodily fluids and compartments dictates the distribution in and ease of elimination from the body. In precipitates and crystals, flow properties can be influenced by a number of factors, such as size and size distribution, shape, habit, polymorph, and porosity, etc.

According to the invention described herein, the “physical state” of a substance, such as a component or a calcium oxalate crystal or calculus is initially defined by whether it is in solid, liquid, or dissolved form. If the substance is a solid, the physical state is further defined by the particle or crystal size, the particle-size distribution, the degree of agglomeration and the habit.

Physical state can further be defined by purity of the solid substance, for example, whether the substance is mineralized, the degree of mineralization, and identity of the minerals. Mechanisms by which impurities can be incorporated in solid substances include surface absorption and entrapment in cracks and crevices, especially in agglomerates and crystals.

Physical state includes whether the substance is crystalline or amorphous. If the substance is crystalline, the physical state is further divided into: (1) whether the crystal matrix includes a co-adduct; (2) morphology, i.e., crystal habit; and (3) internal structure (polymorphism). In a co-adduct, the crystal matrix can include either a stoichiometric or non-stoichiometric amount of the adduct, for example, a crystallization solvent or water, i.e., a solvate or a hydrate.

Non-stoichiometric solvates and hydrates include inclusions or clathrates, that is, where a solvent or water is trapped at random intervals within the crystal matrix, for example, in channels.

A stoichiometric solvate or hydrate is where a crystal matrix includes a solvent or water at specific sites in a specific ratio. That is, the solvent or water molecule is part of the crystal matrix in a defined arrangement. Additionally, the physical state of a crystal matrix can change by removing a co-adduct, originally present in the crystal matrix. For example, if a solvent or water is removed from a solvate or a hydrate, a hole is formed within the crystal matrix, thereby forming a new physical state. Such physical states are referred to herein as dehydrated hydrates or desolvated solvates.

The crystal habit is the description of the outer appearance of an individual crystal, for example, a crystal may have a cubic, tetragonal, orthorhombic, monoclinic, triclinic, rhomboidal, hexagonal, rod, needle, or lathe shape.

The internal structure of a crystal refers to the crystalline form or polymorphism. A given compound may exist as different polymorphs, that is, distinct crystalline species. In general, different polymorphs of a given compound are as different in structure and properties as the crystals of two different compounds. Solubility, melting point, density, hardness, crystal shape, optical and electrical properties, vapor pressure, and stability, etc. all vary with the polymorphic form.

5.2. Preparing Experiments/Arrays and Processing Arrays

The array technology described herein is a high-throughput approach that can be used to generate large numbers (greater than 10, more typically greater than 50 or 100, and more preferably 1000 or greater samples) of parallel small scale samples.

The basic requirements for array and sample preparation and screening thereof are: (1) a distribution mechanism to add components and the medium to separate sites, for example, on an array plate having sample wells or sample tubes; and (2) a screening mechanism to test each sample to detect a change in physical state or for one or more properties. Preferably, one or both of the distribution mechanism and the testing mechanism is automated and controlled by computer software. An automated distribution mechanism can preferably vary at least one addition variable, e.g., the identity of the component(s) and/or the component concentration, more preferably, two or more variables. Such material handling technologies and robotics are well known to those skilled in the art. Of course, if desired, individual components and the medium can be placed at the appropriate sample site manually. This pick and place technique is also known to those skilled in the art. Preferably, the system further comprises a processing mechanism to process the samples after component addition.

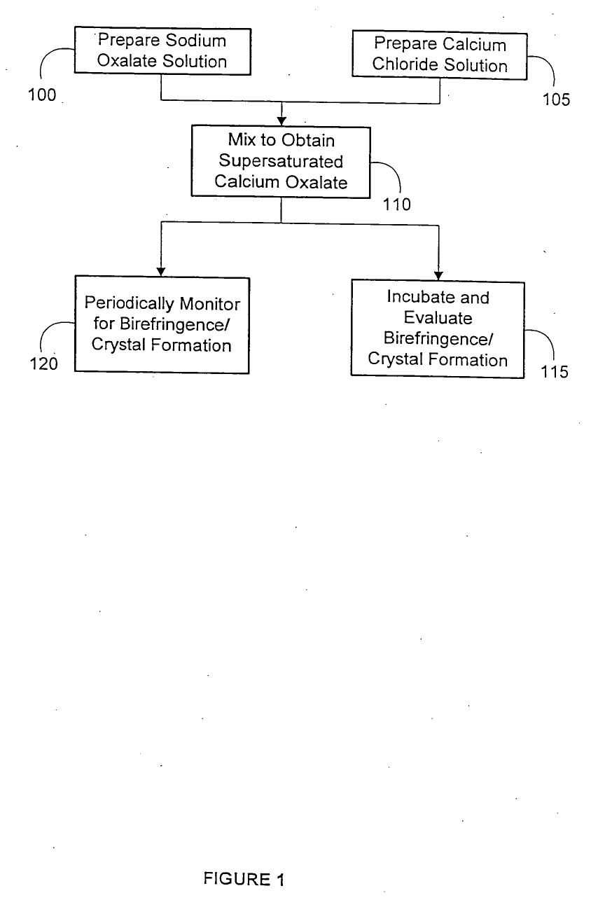

The system also includes means for preparing metastable formulations such as supersaturated solutions. In a preferred design, supersaturated media for nucleating and growing crystals are prepared by mixing two stable components to give rise to a metastable supersaturated solution. As an example illustrated in FIG. 1, a solution of sodium oxalate is mixed with calcium chloride to prepare a solution that is supersaturated in calcium oxalate. In FIG. 1 during step 100, a solution comprising sodium oxalate is prepared. Various additives, i.e., components, may be present in this solution as well. Similarly, during step 105 a solution comprising calcium chloride is prepared. The two solutions are mixed, in each well or location of an array, during step 110 resulting in supersaturated levels of calcium oxalate at a defined time. The mixing may be by mechanical agitation, sonication, stir bars, and the like. This supersaturated mixture may be monitored after incubation for a predetermined duration during step 115. Alternatively, the formation and/or growth of crystals may be examined periodically during step 120.

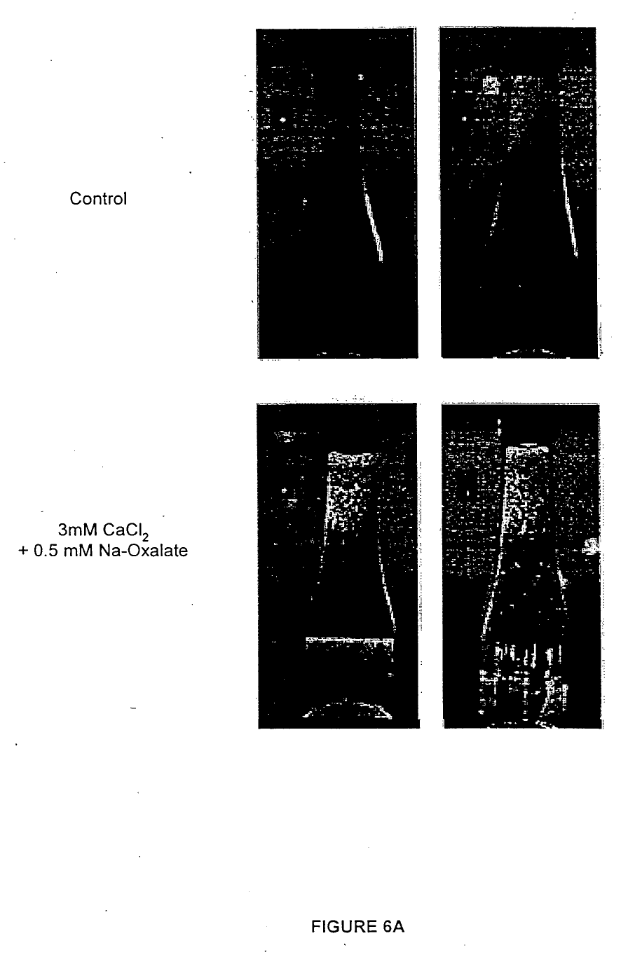

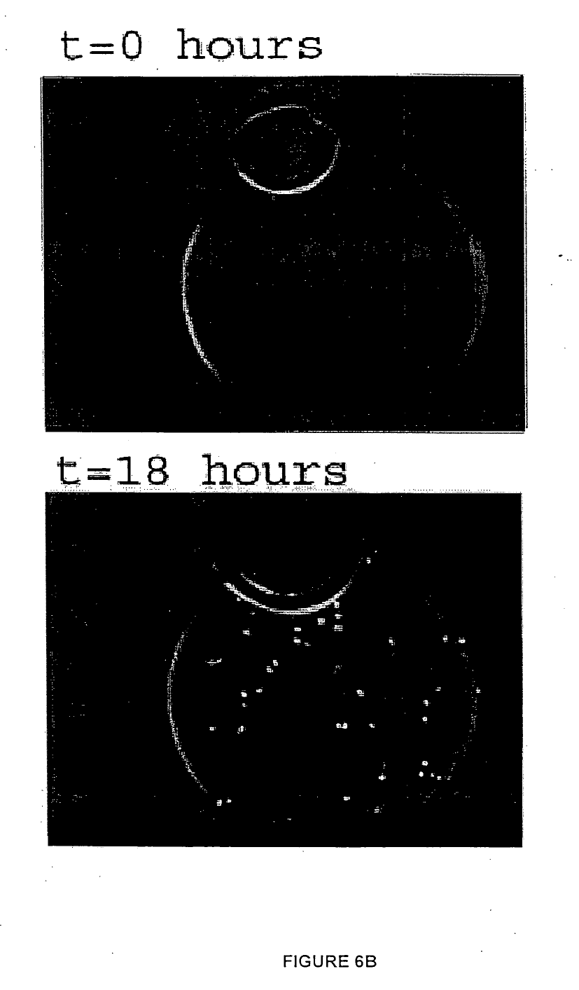

Preferably, the monitoring detects birefringence to indicate the nucleation and/or growth of crystals. Naturally, interfering birefringent components and containers should not provide a signal that overwhelms the desired signal from the crystals. Such monitoring is illustrated in FIGS. 6A & 6B, wherein the sample is photographed through a polarized filter oriented to block incident polarized light on the solutions. Thus, only the optically active components are visible as is seen in the lower panels of FIGS. 6A and 6B. Alternatively, crystal formation, nucleation, or growth may also be tracked by monitoring light scattering, e.g., at 620 nm, due to the formation of a precipitate. Many other techniques for monitoring crystal formation, nucleation, or growth are possible, including observation by humans, filtering the solution, and the like.

Preferably, for high throughput applications, detection of birefringence is used to monitor crystal growth and/or nucleation due to the ease of automating the process by capturing images in a digital camera and processing it with suitable software. Such an implementation allows periodic inspection and intervention by a human operator whenever it is required.

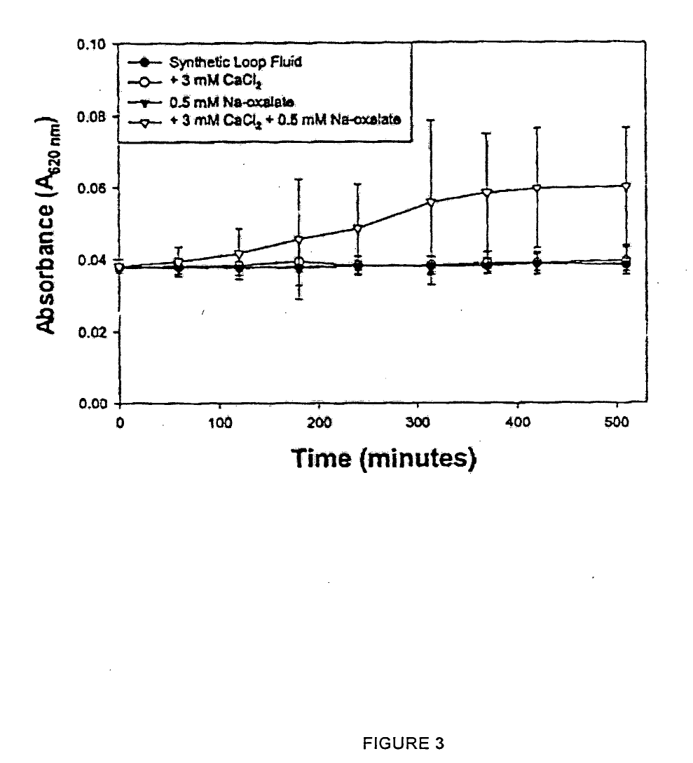

Monitoring samples at 620 nm for turbidity is also possible as is shown in FIG. 3. FIG. 3 illustrates the turbidity, reflecting formation or nucleation of calcium oxalate crystals upon mixing 3 mM calcium chloride with 0.5 mM sodium oxalate. The rapid rise in turbidity for the open triangles in FIG. 3 is in contrast with other fluid preparations, also shown in FIG. 3, such as 3 mM calcium chloride (open circles) or 0.5 mM sodium oxalate (closed triangles) alone, or loop fluid (closed circles).

An array can be prepared, processed, and screened as follows. The first step comprises selecting the medium and component sources, preferably, at one or more concentrations. Preferably, at least one component source can deliver crystalline compounds, or a compound or substance capable of crystallization, such as calcium oxalate. That is, for example, one component source should comprise at least one of: calcium oxalate in undissolved form; calcium oxalate in dissolved form; or the components necessary—in dissolved or undissolved form—to induce formation of calcium oxalate containing solutions, suspensions, or supersaturated preparations. Next, adding the medium and the components to a plurality of sample sites, such as sample wells or sample tubes on one or more sample plates to give an array of unprocessed samples. The array can be processed according to the purpose and objective of the experiment, and one of skill in the art will readily ascertain the appropriate processing conditions. For example, the samples can be processed by heating, cooling, adding additional components, such as acids or bases, stirring, milling, filtering, centrifuging, emulsifying, or by simply allowing the samples to stand for a period of time at a specified temperature, for example, at normal human-body temperature. Each sample in an array can be screened, multiple times if necessary, to determine the presence or absence of crystals or calculi (e.g., calcium oxalate crystals or calculi) and whether crystal growth occurred, and thus determining whether a sample contains one or more compounds or substances that inhibit or modify crystal growth, nucleation, or both, and thereafter testing the same for one or more properties. The data so collected are stored for subsequent data analysis, preferably, by a computer.

Preferably, the automated distribution mechanism used in accordance with the invention can distribute or add components in the form of liquids, solids, semi-solids, gels, foams, pastes, ointments, suspensions, or emulsions. Solids can be in any form, for example, powders, tablets, or pellets. Dispensing may be with the aid of directed deposition of AUTODOSE™, electrostatics as implemented by DELASYS™ or dielectrophoresis developed at Massachusetts Institute of Technology, or other dispensing strategies based on plugs, adhesives and the like.

Arrays preferably include tubes or wells that are clear, or semi-clear to enable measurement of crystal growth and the presence or absence of solids or crystals. Preferably, crystal growth measurements are made by quantitating the increase in total crystal area, crystal long and short axes, and/or other relevant parameters, such as crystal shape, by image capture using a digital camera, e.g., the DVT 544 series digital camera (DVT Corporation, Norcross, Ga.) mounted on a microscope or other optical device. Imaging can be performed using either unpolarized light or polarized light, which could be advantageous for imaging some types of crystals.

In one embodiment, a system where a solid component source, such as a disease-causing-solid source, a solid-component source and a liquid-distribution source, such as a medium source or a liquid-component source, or multiple liquid sources automatically distribute the respective solids or liquids to the sample sites, such as sample wells in a 384 well plate (commercially available, for example, from Millipore, Bedford, Mass.) to give a plurality of samples. The combinations of the medium and various components at various concentrations or combinations are generated using standard software (e.g., Matlab software, commercially available from Mathworks, Natick, Mass.). The combinations thus generated can be downloaded into a spreadsheet, such as Microsoft EXCEL. From the spreadsheet, a work list can be generated for instructing the automated distribution mechanism to prepare an array of samples according to the various combinations generated by the formulating software. The work list can be generated using standard programming methods according to the automated distribution mechanism employed. The use of so-called work lists simply allows a file to be used as the process command rather than discrete programmed steps. The work list combines the formulation output of the formulating program with the appropriate commands in a file format that is directly readable by the automatic distribution mechanism. Preferably, the automated distribution mechanism can deliver multiple amounts of each component.

Automated liquid distribution mechanisms are well known and commercially available, such as the TECAN GENESIS (available from Tecan-US, Research Triangle Park, N.C., USA). Automated solid distribution mechanisms are readily obtained by modifying commercially available robotics systems.

After dispensing is complete the plates can be sealed, if desired, to prevent evaporation or to protect the sample contents from air or light. The sealing mechanism can be a glass plate with an integrated chemically compatible gasket or a variety of plastic or metal foil seals bound to the plate by adhesives or by heating. If tubes are used, the sealing mechanism can be crimp caps, screw caps, corks, non-reactive plugs, and the like.

In a preferred embodiment, a high throughput method for discovering conditions, compounds, or compositions for modifying crystals or a calculus comprising calcium oxalate, comprises preparing seed crystals of calcium oxalate, for instance by obtaining calcium oxalate powder or mechanically fragmenting calcium oxalate crystals or kidney stones or even making seed crystals. Next, the seed crystals are incubated in supersaturated calcium oxalate medium. This medium may have one or more of components for testing. Nucleation and changes in a crystal property (such as crystal growth) are detected by comparing signals obtained from a sample at different times and/or after changing the medium. For example, a signal for a sample can be taken at the beginning of incubation, and then optionally at one or more times during incubation, and then at the end of incubation. The number and time for obtaining signals from or screening a sample will depend on the particular experiment and will be readily apparent to the skilled artisan. These signals or screens of samples may be photographs of birefringent spots, the size of the spots (measured in pixels), calculations based on the pixels and their distribution in a birefringence- or plain light-based image, in-situ Raman spectroscopy to monitor growth or determine the composition of one or more crystals, signals for the concentration of component(s) in the medium, and the like.

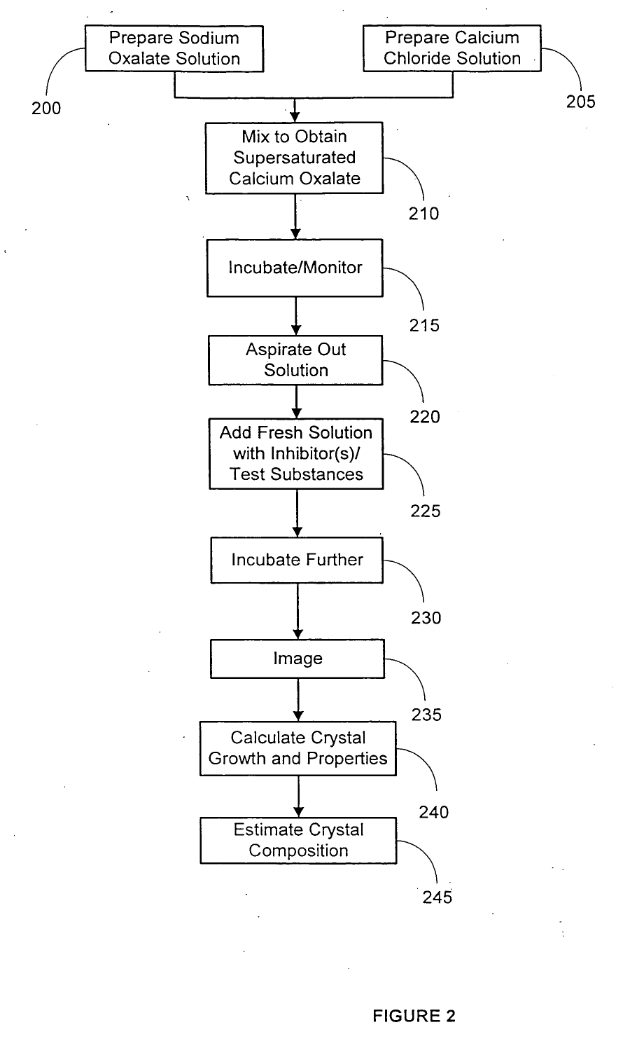

FIG. 2 illustrates one such embodiment. Sodium oxalate and calcium chloride solutions are prepared during steps 200 and 205 respectively. Following mixing during step 210 to obtain supersaturated calcium oxalate medium, also described in FIG. 1, monitoring while incubating during step 215 detects the formation of seed crystals. Next, the medium is removed during step 220 followed by addition of fresh medium containing test components during step 225. The test component(s) are preferably added to the sodium oxalate solution used to make the medium. Alternatively, they could be added in the calcium chloride solution for making the medium, or in a separate solution, or as solids, or even mixed in, or any combination thereof.

The array thus prepared can then be processed according to the design and objective of the experiment. One of skill in the art will readily ascertain the appropriate processing conditions. Processing includes mixing; agitating; heating; cooling; adjusting the pressure; adding additional components, such as acids or bases; stirring; milling; filtering; centrifuging, emulsifying, subjecting one or more of the samples to mechanical stimulation; ultrasound; shock waves; or laser energy; or allowing the samples to stand for a period of time at a specified temperature; for example, normal human-body temperature.

In one embodiment, the array can be processed by heating to a temperature (T1), preferably to a temperature at which the all the solids are completely in solution. The samples are then cooled, to a lower temperature T2, usually for at least one hour. The presence or absence of solids or crystals is then determined.

In another embodiment, the samples can be processed by introducing a nucleation event. Nucleation events include mechanical stimulation and exposure to sources of energy, such as acoustic (ultrasound), electrical, or laser energy. Adding components that decrease the surface energy can also induce nucleation. During cooling, each sample is analyzed for the presence of solid formation. This analysis allows determination of the precipitation or crystallization temperature. Alternatively, samples can be maintained at a constant temperature, e.g., human body temperature, and crystal nucleation and growth can be driven by maintaining supersaturating conditions for the relevant compounds of particular relevance of calcium oxalate.

Advantageously, samples in commercially available micro titer plates can be screened for the presence of solids (e.g., precipitates or crystals) using automated plate readers. Automated plate readers can measure the extent of transmitted light across the sample. Particles (e.g., crystals) in the well or tube scatter incident light leading to a decrease in the amount of light reaching the detector. The decrease in light transmittance (increase in apparent absorbance) is proportional to the aggregate of the number, size and shape of particles (e.g., crystals) contained in the solution. Visual examination of these plates can also be used to detect the presence of solids. In yet another method to detect solids, the plates can be scanned for precipitation by measuring turbidity.

If desired, samples containing solids can be filtered to separate the solids from the medium, resulting in an array of filtrates and an array of solids. For example, the filter plate comprising the suspension is placed on top of a receiver plate containing the same number of sample wells, each of which corresponds to a sample well on the filter plate. By applying either centrifugal or vacuum force to the filter plate over receiver plate combination, the liquid phase of the filter plate is forced through the filter on the bottom of each sample well, into the corresponding sample well of the receiver plate. The appropriated centrifuge is available commercially, for example, from DuPont, Wilmington, Del. The receiver plate is designed for analysis of the individual filtrate samples.

Some processing of arrays is also seen in the method illustrated in FIG. 2. Further incubation of seed crystals during step 230 and imaging during step 235 provide data about the growth and/or nucleation of crystals. The high throughput method of FIG. 2 includes estimating a nucleation and/or growth rate from a number of birefringent spots. An increase in the nucleation rate, or observing little or no crystal growth, ascribable to a component may indicate a therapeutic use for treating diseases, conditions, or disorders associated with calculi or crystal deposition (e.g., nephrolithiasis) by reducing the average size of the crystals, stones or calculi to facilitate excretion. Alternatively, a significant decrease in the nucleation rate ascribable to a component may indicate a therapeutic use for treating diseases, conditions, or disorders associated with calculi or crystal deposition (e.g., nephrolithiasis) by decreasing the rate of stone or calculi formation.

All of the methods described herein can be performed in an iterative manner in order to discover, identify, select, or optimize compounds or substances that achieve a desired result. For example, a first screen comprising an array of several thousand samples may result in 100 hits or samples that appear to contain one or more components that prevent or modify calcium oxalate crystal nucleation, crystal growth, or both. Then, the components designated as hits can be rearrayed (e.g., by changing various conditions, such as component concentrations, media, and the like), and a more focused screen is run. Alternatively, or even simultaneously, solids or crystals from the first screen that were present in “hit” samples (e.g., calcium oxalate seed crystals that exhibit little or no growth in a sample) can be collected (e.g., by filtration), and used to generate one or more samples in a new array in order to determining the strength of the binding between the crystal growth inhibiting component from the original sample and the face of the calcium oxalate crystal. Methods of screening using these samples can be used to test the off-rate (or dissociation) or “stickiness” of an inhibitor identified in earlier screens.

Different temperatures can be used during the solid growth phase. Typically, several distinct temperatures are tested during solid precipitation and crystal nucleation. Temperature can be controlled in either a static or dynamic manner. Static temperature means that a set incubation temperature is used throughout the solid formation process. Alternatively, a temperature gradient can be employed. For example, the temperature is decreased or raised at a constant rate throughout the solid formation

Stand-alone devices employing Peltier-effect cooling and joule-heating are commercially available for use with micro titer plate footprints. A standard thermocycler used for PCR, such as those manufactured by MJ Research or PE Biosystems, can also be used to accomplish the temperature control. The use of these devices, however, necessitates the use of conical vials or conical bottom micro-well plates. If greater throughput or increased user autonomy is required then full-scale systems such as the advanced Chemtech Benchmark Omega 96™ or Venture 596™ would be the platforms of choice. Both of these platforms utilize 96-well reaction blocks made from Teflon™. These reaction blocks can be rapidly and precisely controlled from −70 to 150° C. with complete isolation between individual wells. Also, both systems can operate under an inert atmosphere and utilize chemically inert handling elements. The Omega 496 system has simultaneous independent dual coaxial probes for liquid handling, while the Venture 596 system has 2 independent 8-channel probe heads with independent z-control. Moreover, the Venture 596 system can process up to 10,000 reactions simultaneously. Both systems offer complete autonomy of operation.

For high throughput screening, preferably the temperature used is similar to the physiologic temperature, i.e., about 37° C., or room temperature. However, other temperatures may be used in particular processing steps, e.g., to solubilize a component.

Supersaturation is the thermodynamic driving force for both crystal nucleation and growth and thus is a key variable in processing arrays. Supersaturation is defined as the deviation from thermodynamic solubility equilibrium. Thus the degree of saturation can be controlled by temperature and concentrations and identities of components. In general, the degree of saturation can be controlled in the metastable region, when the metastable limit has been reached, nucleation and growth will be induced. The previously described FIG. 1 provides an approach for controlling saturation and preparing supersaturated solutions.

Crystal nucleation is the formation of a crystal solid phase from a liquid or amorphous phase. Nucleation sets the character of the crystallization process. Primary nucleation can occur by heterogeneous or homogeneous mechanisms. Primary nucleation does not involve existing crystals, but results from spontaneous crystal formation. Primary nucleation can be induced by increasing the saturation over the metastable limit or, when the degree of saturation is between supersaturation and the metastable limit, by introducing a nucleation event. Nucleation events include mechanical stimulation and exposure to sources of energy, such as acoustic (ultrasound), electrical, or laser energy. Adding primary nucleation promoters, that is substances other than the substance to be crystallized, can also induce primary nucleation. Additives that decrease the surface energy of the compound to be crystallized can induce nucleation. A decrease in surface energy favors nucleation, since the barrier to nucleation is caused by the energy increase upon formation of a solid-liquid surface.

Thus, in the current invention, nucleation can be controlled by adjusting the interfacial tension of the crystallizing medium by introducing surfactant-like molecules either by pre-treating the sample tubes or sample wells or by direct addition. The nucleation effect of surfactant molecules is dependent on their concentration and thus this parameter should be carefully controlled. Such tension adjusting additives are not limited to surfactants. Many compounds that are structurally related to the compound to be crystallized can have significant surface activity. Other heterogeneous nucleation inducing additives include solid particles of various substances, such as solid-phase excipients or impurities. Similarly, inorganic crystals on specifically functionalized self-assembled monolayers (SAMs) have also been demonstrated to induce nucleation by Wurm, et al., 1996, J. Mat. Sci. Lett. 15:1285 (1996). Nucleation of organic crystals such as 4-hydroxybenzoic acid monohydrate on a 4-(octyldecyloxy)benzoic acid monolayer at the air-water interface has been demonstrated by Weissbuch, et al. 1993 J. Phys. Chem. 97:12848 and Weissbuch, et al., 1995 J. Phys. Chem. 99:6036. Nucleation of ordered two dimensional arrays of proteins on lipid monolayers has been demonstrated by Ellis et al., 1997, J. Struct. Biol. 118:178.

Secondary nucleation involves treating the crystallizing medium with a secondary nucleation promoter. Direct seeding of samples with a plurality of nucleation seeds in various physical states provides a means to induce formation of different crystal forms. In one embodiment, particles are added to the samples. In another, nanometer-sized crystals (nanoparticles) are added to the samples.

These and other seeding techniques are referenced in FIG. 2 as well. Birefringence based monitoring includes illuminating crystals with polarized light followed by photographing the illuminated crystals via a cross polarizer to detect birefringent crystals and detecting a plurality of crystal spots from a photograph of the illuminated crystals. The number of detected spots over time provides an estimate of the nucleation rate.

As discussed above, particle size is very relevant to the clinical aspects of disease-causing solids or crystals. Particle size and size distribution often dictates the pathology of disease-causing solids. Particulate matter, produced by precipitation or crystallization, has a distribution of sizes that varies in a definite way throughout the size range. Particle- and crystal-size distribution is generally expressed as a population distribution relating to the number of particles at each size. In calcium oxalate calculi, particle and crystal size distribution have very important clinical aspects. For example, smaller particles are more easily eliminated from the body and have higher surface-to-volume ratio that allows easier dissolution in bodily fluids. The particle size of substances formed by crystallization or precipitation from solvents or other fluids, such as bodily fluids, is influenced by a number of factors, such as nucleation, number of nucleation sites, degree of saturation of the substance, solubility of the substance. The structure of the formed precipitate or crystals also influences particle size and size distribution. Structural properties, such as habit, polymorphism, porosity, and composition can affect crystal size in many ways, such as providing additional nucleation sites and affecting the growth rate.

The photograph of the illuminated crystals, e.g., obtained in step 235 of FIG. 2, may be further analyzed to estimate a number of pixels corresponding to a crystal spot. Comparing the number of pixels to an initial number of pixels for the same crystal reveals a change in the number of pixels. This change is useful for estimating any changes in crystal size, with no change or a reduction in an average crystal size ascribable to a component suggesting a therapeutic use in treating diseases, conditions, or disorders associated with calcium oxalate crystals or calculi (e.g., nephrolithiasis) by enabling easier excretion of calcium oxalate. TABLE 1 below provides an example of crystal size and distribution estimates obtained in a similar manner.

TABLE 1

| Crystal | | | Change in | |
|---|---|---|---|---|
| Number | Initial Area | Final Area | Area | % Change |
| 1 | 3 | 21 | 18 | 0.857 |
| 2 | 7 | 68 | 61 | 0.897 |
| 3 | 5 | 60 | 55 | 0.917 |
| 4 | 6 | 66 | 60 | 0.909 |
| 5 | 5 | 48 | 43 | 0.896 |
| 6 | 2 | 62 | 60 | 0.968 |
| 7 | 2 | 61 | 59 | 0.967 |
| 8 | 4 | 36 | 32 | 0.889 |
| 9 | 3 | 7 | 4 | 0.571 |
| 10 | 4 | 8 | 4 | 50.0 |
| 11 | 3 | 25 | 22 | 0.88 |
| 12 | 4 | 130 | 126 | 0.969 |
| 13 | 1 | 40 | 39 | 97.5 |
| 14 | 5 | 51 | 46 | 0.902 |
| Average | | | 44.92857143 | 0.864 |
| Standard | | | 30.87559227 | 0.145 |
| Deviation | | | | |

Other properties of crystals may also be obtained from the pixel measurements in step 240 of FIG. 2. The photograph of the illuminated crystals also allows an estimate of a long and a short dimension among many possible measures. These reflect the habit of the crystal spot and may be used to predict crystals that are likely to be excreted more easily. For instance, small and even shaped crystals are more likely to be excreted than spiny crystal shapes, thus permitting high throughput screening for promising therapeutic candidates.

Samples can be incubated for various lengths of time. Since physical-state changes, particularly crystallizations, can occur as a function of time, it is advantageous to examine arrays over a range of times.

The charge of the compound being precipitated or crystallized can determine the nature of the solid phase that is generated. The pH can be modified by the addition of inorganic and/or organic acids and bases, additional crystallization additives such as small molecules, macromolecules, and solvents.

The use of different solvents or mixtures of solvents can inhibit or promote physical-state changes and influence the type of physical state change, such as whether crystals or a precipitate is generated. Solvents may influence and direct the formation of the solid phase through electrostatic properties, charge distribution, molecular shape and flexibility, and pH. Solvents accepted for use in drug manufacture are preferred for use in the arrays of the invention. Various mixtures of those solvents can be used. Solvents include, but are not limited to, aqueous-based solvents such as water, aqueous acids, bases, salts, and buffers or mixtures thereof and organic solvents, such as protic, aprotic, polar or non-polar organic solvents. Notably, some components in a solvent of choice for testing may be added by rapidly diluting them into the supersaturated calcium oxalate medium.

The invention encompasses use of human and/or animal and/or synthetic (also referred to as synthetic loop fluid) urine as a solvent for one or more component. Moreover, the urine may be from patients or animals exhibiting nephrolithiasis or other diseases, conditions, or disorders associated with calcium oxalate crystal deposits or calculi, people or animals susceptible to nephrolithiasis or other diseases, conditions, or disorders associated with calcium oxalate crystal deposits or calculi, or normal urine. As noted previously, components include fractions prepared from urine, for instance, by chromatographic techniques like HPLC. Therefore, a solvent (as an alternative to being a non-urine liquid carrying a component) may be a solution comprising urine. In addition, a non-urine solvent carrying a component may be diluted into a solution comprising urine.

The concentration of the components can influence, promote, or inhibit changes in physical state, for example, whether a crystal or a precipitate is formed. The temperature at which crystallization is thermodynamically possible is generally a strong function of concentration. Crystal-growth rate and the rate of nucleation of crystals increases with increasing concentration, which can affect crystal habit. For example, rapid growth must accommodate the release of the heat of crystallization. This heat effect is responsible for the formation of dendrites during crystallization. The macroscopic shape of the crystal is profoundly affected by the presence of dendrites and even secondary dendrites. Another effect of concentration of components is the chemical composition of the crystal or precipitate formed. For example, a concentrated solution may first form crystals of the hemihydrate when precipitated from aqueous solution at high temperature. The dihydrate may, however, be the first to form when starting with a dilute solution.

5.3. Analyzing the Arrays

After processing, the samples are analyzed to detect those samples wherein a change in physical state occurred. In one embodiment, the sample's filtrate can be analyzed to detect or measure concentrations of dissolved substances, for example, the concentration of a dissolved disease-causing solid. Solubility can be analyzed using devices, such as UV-Vis spectroscopy (using plate-based readers known to those skilled in the art, an example of which is the SPECTRAMAX PLUS (available from Molecular Devices, Sunnyvale, Calif.)), GC, HPLC, and LC-MS. In the case of GC, HPLC, and LC-MS, an automated pipetting station is used for sample introduction, for example, the GENESIS from Tecan or any of several devices sold by Gilson, Middleton, Wis. USA.

In another embodiment, solid materials present in the samples are analyzed, such as solid calcium oxalate. Suitable methods of analysis include, but are not limited to, absorbance measurements, preferably at about 620 nm, imaging in polarized and normal light, automated image analysis to measure total light output and heterogeneity of light intensity as a function of position on the plate or other vessel to determine the number of particles, hemocytometry to estimate the number of particles/crystals, UV spectrometry, visible light spectrometry, x-ray diffraction crystallography, such as single crystal or powder x-ray diffraction; second harmonic generation (SGH); microscopy; photomicrography; thermal methods of analysis, such as thermogravimetric analysis (TGA) and differential thermal analysis (DTA); electron microscopy; infrared spectroscopy; and analytical methods requiring dissolution of the solid, such as ultraviolet spectroscopy, nuclear magnetic resonance (NMR) spectroscopy; polarography; gas chromatography; and high-pressure liquid chromatography (HPLC). Solids can also be analyzed by subjecting them to energy forms, such as laser, ultrasound, or shock waves to determine their resistance thereto.

Other analytical devices that can be used with the methods and arrays of the invention include pH sensors, ionic strength sensors, optical spectrometers, devices for measuring turbidity, calorimeters, infrared spectrometers, polarimeters, radioactivity counters, conductivity measurers, and heat of dissolution measurers.

Data collection and storage preferably, are performed by computers using the appropriate software. Such computers and software are readily chosen by one of skill in the art. The data are typically collected and stored directly from the analytical equipment using software provided by the instrumentation's manufacturer or custom software. The data set can then be downloaded to a database for analysis.

Data analysis can be performed using visualization software, such as SPOTFIRE (commercially available from Spotfire, Inc., Cambridge, Mass.). The visualized data can be analyzed directly to arrive at optimized conditions, compounds, or compositions. Or the data can be processed through data mining algorithms so as to optimize the ability of scientific personnel to detect complex multi-dimensional interactions or lack of interactions between components or to conduct future experiments to optimize the formulations. Examples of suitable data-mining software include, but are not limited to, SPOTFIRE; MATLAB (Mathworks, Natick, Mass.); STATISTICA (Statsoft, Tulsa, Okla.). All resulting analysis files are stored on a central file server, i.e., a database, for ready access.

The screening of arrays may be directed to detecting substances that can inhibit crystal growth or inhibit nucleation of crystals. Furthermore, the screening can identify substances that irreversibly inhibit growth so that removal of the substance does not result in resumption in crystal growth and/or nucleation. FIGS. 6 & 7 illustrate such methods.

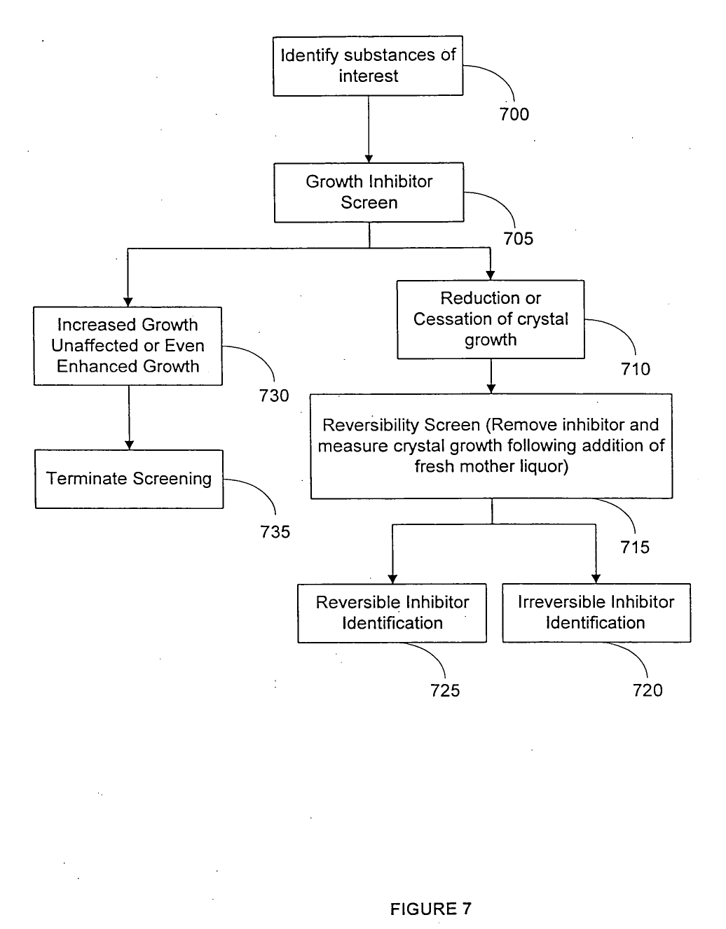

In FIG. 7, during step 700 one or more substances of interest are identified. Preferably, but not as a requirement, these substance are known to be safe for administration as drugs at the concentrations and amounts being tested. The identified substances, added in solutions of calcium chloride or sodium oxalate, are subjected to screening via a high-throughput growth inhibitor screen during step 705. Accordingly, growth of crystals in supersaturated calcium oxalate medium is observed in the presence or absence of identified substances. Such growth may be of seed crystals or may include nucleation of crystals as well as growth. Comparison of the growth of calcium oxalate crystals in the presence of various levels, including absence of, the identified substances (and their various combinations) reveals significant inhibition of calcium oxalate crystal growth during step 710. As discussed later, at this stage, changes in habit of calcium oxalate crystals may also be detected, including via automated image analysis.

Washing the crystals and replacing the medium having an inhibitor of crystal growth by medium without the inhibitor during step 715 implements a high-throughput reversibility screen to identify substances, during step 720, that correspond to irreversible inhibition of calcium oxalate crystal growth. The inhibition of crystal growth need not result in complete cessation of crystal growth, and instead, may reveal a lower rate of growth than expected. The observed rate of growth may be greater than that observed in the presence of the inhibitor while being less than the rate expected in the absence of the inhibitor. Identification, during step 725, of substances exhibiting reversible inhibition of crystal growth may result in treatments that potentially require ongoing dosing to reduce crystal growth.

Notably, substances that enhance calcium oxalate crystal growth or do not acceptably inhibit or reduce it are also identified by the high-throughput growth inhibitor screen during steps 705 and 730. In a preferred embodiment, but not as a requirement for practicing the invention, such substances are not screened further as shown in step 735.

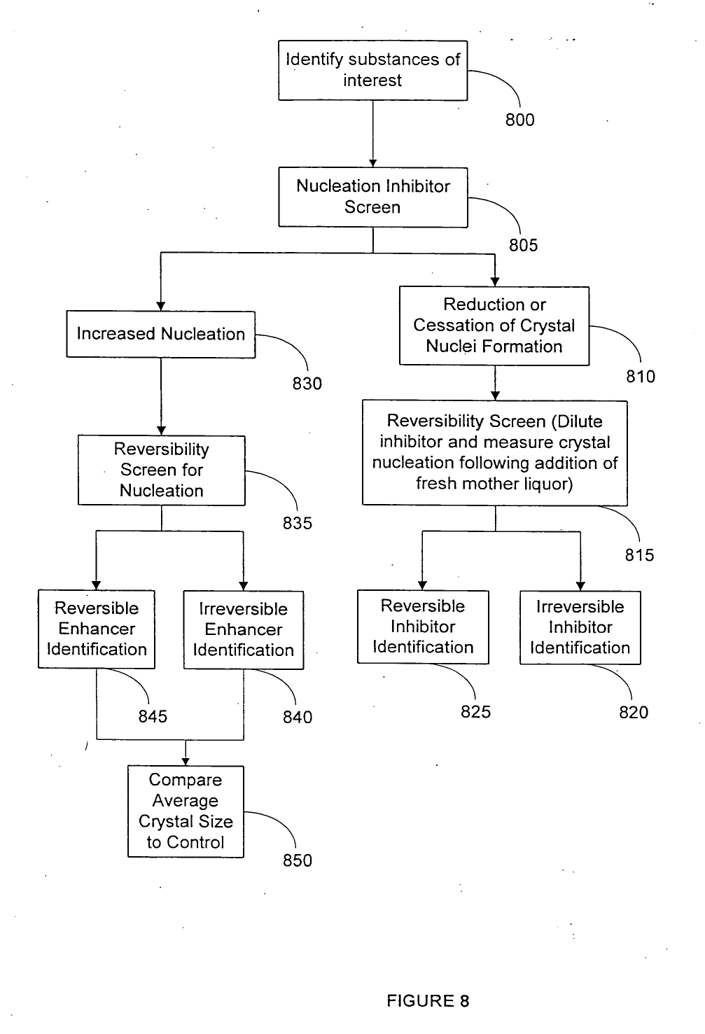

Similarly, in FIG. 8, during step 800 one or more substances of interest as putative inhibitors or enhancers of calcium oxalate crystal nucleation are identified. Preferably, but not as a requirement, these substances are known to be safe for administration as drugs at the concentrations and amounts being tested. The identified substances, added in solutions of calcium chloride or sodium oxalate, are subjected to screening via a high-throughput nucleation inhibitor screen during step 805. Accordingly, growth of crystals in supersaturated calcium oxalate medium is observed in the presence or absence of identified substances. Such growth may be of seed crystals or may include nucleation of crystals as well as growth. Comparison of the growth of calcium oxalate crystals in the presence of various levels, including absence of, the identified substances (and their various combinations) reveals significant inhibition of calcium oxalate crystal nucleation during step 810. Such reduction may be detected, for instance, by examining, following or during incubation, a sample for the number and rate of development of birefringent deposits.

FIG. 6 shows an example of a birefringent image showing the location and formation of calcium oxalate crystals following an eighteen-hour incubation. The top panel in FIG. 6 shows the supersaturated calcium oxalate medium illuminated by polarized light and viewed through a cross polarizer to detect birefringence. Due to the absence of any crystals the entire field is dark. On the other hand, after eighteen hours, as shown in the lower panel of FIG. 6, several specks of light are visible due to rotation of light by birefringent crystalline deposits visible as the specks. Automated image analysis can readily track the number and the rate of formation of such spots.

Diluting the medium having an inhibitor of crystal nucleation by medium without the inhibitor during step 815 implements a high-throughput nucleation reversibility screen to identify substances, during step 820, that correspond to irreversible inhibition of calcium oxalate crystal nucleation. The inhibition of crystal nucleation need not result in complete cessation of crystal nucleation, and instead, may reveal a lower rate of nucleation than expected. The observed rate of nucleation may be greater than that observed in the presence of the inhibitor while being less than the rate expected in the absence of the inhibitor. Identification, during step 825, of substances exhibiting reversible inhibition of crystal nucleation may result in treatments that potentially require ongoing dosing to reduce nucleation of crystals.

Notably, substances that enhance calcium oxalate crystal nucleation are also identified by the high throughput nucleation inhibitor screen during steps 805 and 830. In a preferred embodiment, but not as a requirement for practicing the invention, such substances are also screened further as shown in steps 835-845 to identify reversible and irreversible enhancers of nucleation using the high-throughput nucleation reversibility described for step 815. Such nucleation enhancers are of possible therapeutic significance since they may increase the number of crystals while reducing the average size of individual crystals. It is easier, and arguably normal, to excrete small crystals of calcium oxalate than to pass large kidney stones. Accordingly, during step 850 the average size of crystals is compared to that without the presence of nucleation enhancer to identify desirable nucleation enhancers from a therapeutic perspective.

In another aspect, the invention encompasses a high-throughput inhibitor-inhibitor screen to identify inhibitors of inhibitors of crystal nucleation and/or growth. In the case of calcium oxalate crystals in urine and other bodily fluids, it is expected that there are naturally present inhibitors of calcium oxalate crystal nucleation and/or growth. Under such a model in disease conditions of interest formation of kidney stones results from inhibition of naturally present inhibitors of calcium oxalate crystals.

The high-throughput inhibitor-inhibitor screen evaluates growth of calcium oxalate crystals in supersaturated calcium oxalate medium formed from urine from individuals and/or animals known to be susceptible to stone formation (hereinafter “susceptible medium”) and normal individuals and/or animals (hereinafter “resistant medium”) and/or synthetic urine (hereinafter “synthetic medium”). Accordingly, mixing in resistant medium inhibits calcium oxalate crystal growth and/or nucleation in synthetic and susceptible media. Moreover, substances, added in solutions of calcium chloride or sodium oxalate, can be tested to identify inhibitors of inhibitors of resistant medium by screening for substances that make susceptible medium (or synthetic medium) more resistant to calcium oxalate crystal growth and/or nucleation. Similarly, potential disease causing agents/fractions isolated from susceptible media that make resistant medium susceptible to calcium oxalate crystal growth and/or nucleation, are readily identified by the high-throughput inhibitor-inhibitor screen.

In yet another aspect of the invention, identity of susceptible medium along with any identified disease-causing agents/fractions prepared therefrom is recorded in a database. Similarly, the various identified substances in the throughput growth inhibitor, high-throughput reversibility, high throughput nucleation inhibitor, high-throughput nucleation reversibility, and high-throughput inhibitor-inhibitor screens are recorded in a database. This information is then used to implement virtual screens such as predicting individuals susceptible to formation of kidney stones to provide treatment prior to formation of the actual stone based on the level and presence of particular disease-causing agents/fractions of interest. Other virtual screens include evaluating a substance for suitability in experiments to identify better agents, doses, or formulations for preventing or ameliorating the effects of nephrolithiasis. Further details of informatics for handling, storing, and using the results of high throughput experiments is provided in U.S. patent application Ser. No. 10/103,983, filed Mar. 22, 2002 and U.S. patent application Ser. No. ______, filed May 10, 2002 (attorney docket no. 10436-035-999), each of which is incorporated herein in its entirety by reference.

5.3.1. Analysis or Detection Systems

In certain embodiments, after processing solids, such as solid calcium oxalate containing samples, can be detected and analyzed. There are several general methods applicable. In the preferred embodiment, so-called machine vision technology is used. Specifically, images are captured by a high-speed charge-coupled device (CCD) camera that has an on-board signal processor. This on-board processor is capable of rapid processing of the digital information contained in the images of the sample tubes or sample wells. Typically, two images are generated for each location of the well that is being analyzed. These two images differ only in that each is generated under different incident light polarization. Differences in these images due to differential rotation of the polarized light indicate the presence of crystals. For wells that contain crystals, the vision system determines the number of crystals in the well, the exact spatial location of the crystals within the well (e.g., X and Y coordinates) and the size of each crystal. In addition to total crystal size, measured as total pixel number, the aspect ratio of the crystal, which directly corresponds to crystal habit can be determined.

The use of on-line machine vision to determine both the absence/presence of crystals as well as detailed spatial and morphological information has significant advantages. Firstly, this analysis provides a “filtering” means to reduce the number of samples that will ultimately undergo in-depth analysis. Secondly, the spatial information gathered on the locations of crystals is critical to the efficiency in which the in-depth analyses can be performed. This information allows for the specific analysis of individual crystals that are two to four orders of magnitude smaller than the wells that they are contained in. Those wells (reservoirs or sites in the array) identified to contain crystalline or other specific solids can then be further analyzed.

Crystallinity can be assessed automatically using plate readers with polarized filter apparatus to measure the total light to determine crystal birefringence. Plate readers are commercially available. It is also possible to monitor turbidity or birefringence dynamically throughout the crystal forming process. Precipitation, which is indicative of crystal formation, is monitored by optical density, which can be visually or spectrophotometrically determined by turbidity. Crystallinity is determined by birefringence, which distinguishes crystals from amorphous material; crystals turn polarized light, while amorphous materials absorb the light. True polymorphs, solvates, and hydrates, are tested by x-ray Powder Diffraction (XRPD) (angles of diffracted laser light can be used to determine whether true polymorph(s) have been formed). Different crystalline forms are determined by differential scanning calorimetry (DSC) and Thermographic Gravimetric (TG) analysis.

The x-ray crystallography technique, whether performed using single crystals or powdered solids, concerns structural analysis and is well suited for the characterization of the chemical nature of identified crystals, including but not limited to identification of salt composition, polymorphs, and solvates as well as distinguishing amorphous from crystalline solids. In the most favorable cases, it can lead to a complete determination of the structure of the solid and the determination of the packing relationship between individual molecules in the solid. Single crystal x-ray diffraction is the preferred analytical technique to characterize crystals generated according to the arrays and methods of the invention. The determination of the crystal structure requires the determination of the unit-cell dimensions and the intensities of a large fraction of the diffracted beams from the crystal.

The first step is selection of a suitable crystal. Crystals should be examined under a microscope and separated into groups according to external morphology or crystal habit. For a complete study, each crystal of a completely different external morphology should be examined.

Once the crystals have been separated according to shape, the best crystal of the first group should be mounted on a goniometer head with an adhesive such as glue.

The unit cell dimensions are then determined by photographing the mounted crystal on a precession camera. The unit cell parameters are determined from the precession photograph by measuring the distance between the rows and columns of spots and the angle between a given row and column. This is done for three different orientations of the crystal, thus allowing determination of the unit cell dimensions.

The intensities of the diffracted radiation are most conveniently measured using an automated diffractometer that is a computer-controlled device that automatically records the intensities and background intensities of the diffracted beams on a magnetic tape. In this device a detector intercepts the diffracted beam with the intensity recorded electronically.

The diffraction data are then converted to electron density maps using standard techniques, for example, the DENZP program package (Otwinowski, et al., Methods in Enzymology 276 (1996)). Software packages, such as XPLOR (A. Brunger, X-PLOR Manual, Yale University), are available for interpretation of the data. For more details, see Glusker, J. P. and Trueblood, K. N. Crystal Structure Analysis”, Oxford University Press, 1972.

X-ray Powder Diffraction can also be used. The method that is usually used is called the Debye-Scherrer method (Shoemaker and Garland, 1962). The specimen is mounted on a fiber and placed in the Debye-Scherrer powder camera. This camera consists of an incident-beam collimator, a beam stop, and a circular plate against which the film is placed. During the recording of the photograph, the specimen is rotated in the beam. Because the crystallites are randomly oriented, at any given Bragg angle, a few particles are in diffracting position and will produce a powder line whose intensity is related to the electron density in that set of planes.

This method, along with precession photography, can be used to determine whether crystals formed under different conditions are polymorphs or merely differ in crystal habit. To measure a powder pattern of a crystal or crystals on a Debye-Scherrer camera, one grinds the sample to a uniform size (200-300 mesh). The sample is then placed in a 0.1- to 0.5-mm-diameter glass capillary tube made of lead-free glass. Commercially made capillary tubes with flared ends are available for this purpose. The capillary tube is placed on a brass pin and inserted into the pin-holder in a cylindrical Debye-Scherrer powder camera. The capillary tube is aligned so that the powdered sample remains in the x-ray beam for a 360° rotation. Film is then placed in the camera, and the sample is exposed to CuK_{α} x-rays. The film is then developed and the pattern is compared to the pattern from other crystals of the same substance. If the patterns are identical the crystals have the same internal structure. If the patterns are different, then the crystals have a different internal structure and are polymorphs.

Symmetry lowering in host-additive systems (crystals incorporating guest molecules, e.g., solvates), such as a loss of inversion, glide, or twofold screw symmetry, which would introduce polarity into the crystal, can be probed by non-linear optical effects, such as second harmonic generation, which is active in a noncentrosymmetric crystalline forms. For a comprehensive review on second harmonic generation see Corn et al., 1994 Chem. Rev. 94:107-125.

Microscopy and photomicrography methods of analysis involve observation of crystals and physical-state changes of crystals under a microscope (Kuhuert-Brandstatter, 1971). Crystals are usually placed on a microscope slide and covered with a cover slip. However, sometimes a steel ring with input and output tubes is used to control the atmosphere. The microscope slide is often placed on a “hot stage,” a commercially available device for heating crystals while allowing observation with a microscope. The heating rate of crystals on a hot stage is usually constant and controlled with the help of a temperature programmer.

Crystals are often photographed during heating. Photography is helpful because for solid-state changes taking weeks to complete it is sometimes difficult to remember the appearance of a crystal during the entire change.

The following types of behavior are of particular interest to the solid-state chemist:
1. The loss of solvent of crystallization.
2. Sublimation of the crystal—the crystal slowly disappears and condenses on the cover slip.
3. Melting and re-solidification, indicating a phase change (polymorphic transformation) or solid-state change.
4. Chemical reaction characterized by a visible change in the appearance of the crystal.

The detection of loss of solvent of crystallization and phase or polymorphic transformations is important to the solid-state chemist, since crystals exhibiting this behavior can have different reactivity and different bioavailability.

Thermal analysis generally refers to any method involving heating the sample and measuring the change in some physical property. The most important thermal methods for the study of solid-state chemistry are thermogravimetric analysis (TGA) and differential thermal analysis (DTA). Thermogravimetric analysis involves measuring the change in the mass of the sample as the temperature is changed. Differential thermal analysis involves measuring the difference between the temperature of the sample and a reference compound as the temperature is changed, and provides information on the enthalpy change of various solid-state processes.

Thermogravimetric Analysis (TGA) involves the measurement of the change in mass with temperature and is often used to study the loss of solvent of crystallization or other solid→solid+gas changes. In studies of solid-state chemistry, TGA is usually performed in one of two modes: isothermal or dynamic. In the isothermal mode, the temperature is constant, while in the dynamic mode the temperature is raised at a constant rate. There are a number of factors that affect TGA curves, including the heating, atmosphere, crystal size of the sample, nature of the change, treatment of the sample, and thermal conductivity of the sample. The affect of the heating rate has been extensively studied (Wendlandt, 1974). In general, as the heating rate is increased, the starting temperature of the thermal event (Tᵢ) increases. However, this condition can sometimes be corrected by decreasing the sample size. The atmosphere can have a dramatic effect on the TGA curve. For example, an atmosphere containing the product gas can increase Tᵢ or stop the change completely. In addition, the atmosphere can change the course of the change, particularly if the atmospheric gas reacts with either the products or the reactant. In general, the crystal size of the sample has a predictable effect on the TGA curve. The smaller the crystal size, the faster the physical change and the lower the value of Tᵢ. This is because the small crystals have relatively large surface areas, and more rapid escape of the product gas is allowed. Obviously, the nature of the change affects Tᵢ. The Tᵢ is lower for the more facile physical changes. In addition, the extent of compression of the sample will affect the Tᵢ. For example, increased compression will increase Tᵢ since the product gas will have fewer opportunities to escape. Finally, the thermal conductivity of the sample will influence Tᵢ and could lead to anomalous effects if the temperature of the sample is not uniform. The rates of physical changes of the type solid→solid+gas can be determined using TGA. Obviously, TGA traces can be used to determine the rate of the physical change and the rate law governing the physical change.

Dynamic TGA has also been used to determine the rates of solid→solid+gas physical changes. However, in general, the kinetic data obtained should be substantiated by other data before it is considered absolutely correct. It should be noted that the use of dynamic TGA to study kinetics has been criticized.

Isothermal thermogravimetric analysis has been used extensively to study the desolvation of crystal solvates.

Differential Thermal Analysis (DTA) is a method in which the temperature of the sample (Tₛ) is compared to the temperature of a reference compound (Tᵥ) as a function of increasing temperature. Thus, a DTA thermogram is a plot of ρT=Tₛ−Tᵥ (temperature difference) versus T. The endotherms represent processes in which heat is absorbed, such as phase transitions and melting. The exotherms represent processes such as chemical reactions where heat is evolved. In addition, the area under a peak is proportional to the heat change involved. Thus, this method with proper calibration can be used to determine the heats γρH) of the various processes, the temperatures of processes such as melting, Tₘ, can be used as an accurate measure of the melting point.

There are a number of factors that can affect the DTA curve, including heating rate, atmosphere, the sample holder and thermocouple location, and the crystal size and sample packing. In general, the greater the heating rate the greater the transition temperature (i.e., Tₘ). An increased heating rate also usually causes the endotherms and exotherms to become sharper. The atmosphere of the sample affects the DTA curve in much the same way it affects the TGA curve. If the atmosphere were one of the reaction products, then increases in its partial pressure would slow down the reaction. The shape of the sample holder and the thermocouple locations can also affect the DTA trace. Thus, it is a good idea to only compare data measured under nearly identical conditions. As with TGA, the crystal size and packing of the sample has an important influence on all reactions of the type solid→solid+gas. In such reactions, increased crystal size (thus decreased surface area to volume ratio) usually decreases the rate of the reaction and increases the transition temperature.

DTA has been used to study the kinetics of solid-state physical changes but, like the TGA methods, this approach has been criticized and results of kinetic studies by DTA should probably be checked by other methods before they are considered reliable.

An important type of differential thermal analysis is differential scanning calorimetry (DSC). Differential Scanning Calorimetry refers to a method very similar to DTA in which the AH of the reactions and phase transformations can be accurately measured. A DSC trace looks very similar to a DTA trace, and in a DSC trace the area under the curve is directly proportional to the enthalpy change. Thus, this method can be used to determine the enthalpies of various processes (Curtin et al., 1969).

Electron microscopy is a powerful tool for studying the surface properties of crystals. High-resolution election microscopy can be used to visualize lattice fringes in inorganic compounds, but its usefulness for visualization of lattice fringes in organic compounds is so far unproven. Nevertheless, electron micrographs of organic crystals allow the examination of the crystal surface during physical or chemical change. Electron microscopy is particularly useful for studying the effects of structural imperfections and dislocations on solid-state organic physical or chemical changes. For example, the surface photooxidation of anthracene is obvious from electron micrographs taken at a magnification of 10,000 (Thomas, 1974). Even more interesting is the use of electron microscopy, sometimes in conjunction with optical microscopy, to study the effects of dislocations and various kinds of defects on the nucleation of product phase during a solid state physical change.

Electron microscopy is also quite useful for the studies of the effect of crystal size on desolvation physical changes. Electron micrographs have significantly more depth of field than optical micrographs, so that the average crystal size can be more easily determined using them.

The particular array members that give rise to the formation of solids may also be analyzed through the use of laser or ultrasound generated shock waves. Ultrasound lithotripsy of renal stones was first reported in 1977. Lithotripsy is a process involving the fragmentation of stone through the use of high-energy shock waves generated by a high-voltage energy source. Shock wave sources such as electrohydraulic, piezoceramic, or electromagnetic devices have been used to fragment gallbladder stones. Fauci et al., Harrison's Principle of Internal Medicine, 14th ed., McGraw-Hill Health Professions Division, 1998. The same shock wave sources may be adapted for use in the present invention.

Fragmentation of the solid materials that formed from the arrays may also be achieved by subjecting stones and other solid materials to laser-generated shock waves. The shock waves produced by lasers generally allow safe fragmentation of urinary calculi compared to the laser-based thermal fragmentation of stones. For clinical applications, laser-based fragmentation method requires that the shock wave energy exceed the stone's tensile strength. H. D. Fair and I. Fensel used a laser induced shock wave in 1968 instead of relying on laser thermal effect to fragment stones using a Q-switched Nd-YAG laser. Laser sources that provide short pulses generally tend to generate significantly less thermal effect than continuous wave lasers. The reduced thermal effect leads to less tissue damage. Various types and brands of continuous or pulsed lasers are commercially available (Coherent, Lambda-Physik, Spectra-Physics, etc.) and their wavelength ranges vary from the infrared to ultraviolet. Femtosecond lasers are also now widely available, in addition to the traditional nanosecond and picosecond pulsed lasers. The laser power or power density may be adjusted accordingly to determine the threshold required for fragmentation.

A holmium-YAG laser having a wavelength at 2100 nm (near infrared) has been used to fragment stones successfully. The fragmentation is based on the energy absorption and water vaporization at the stone's surface and its pores. The color and composition of the stones have been found to be irrelevant. The first commercially produced lithotriptor, HM3, was introduced in 1984. Nowadays, most non-passable upper urinary tract stones are treated with extracorporeal shock wave lithotripsy. There are now about 30 different Extracorporeal Shock Wave lithotripsy devices available and electrohydraulic energy source is one of the most widely used sources.

This invention also encompasses the use of Raman and Infrared spectroscopy for analysis of solids, one advantage being that it can be performed without sample dissolution. The infrared and near infrared spectrum are extremely sensitive to structure and conformation. The method involves grinding the sample and suspending it in Nujor or grinding the sample with KBr and pressing this mixture into a disc. This preparation is then placed in the near infrared or infrared sample beam and the spectrum recorded.

Raman and Infrared spectroscopy are also useful in the investigation of polymorphs in the solid state. The provisional patent application 60/318,138 filed on Sep. 7, 2001, describing a binning procedure based on Raman spectroscopy to estimate the number of forms of a substance, is incorporated herein by reference in its entirety. Thus, for instance, the nature and number of various forms of calculi responsible for kidney disease or disorder may be evaluated by Raman spectroscopy.

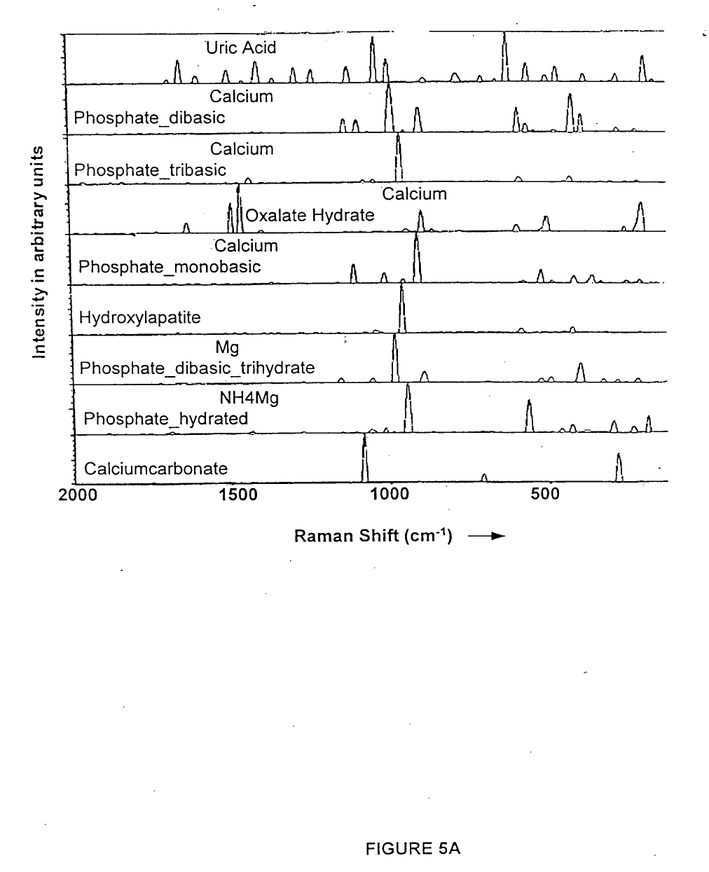

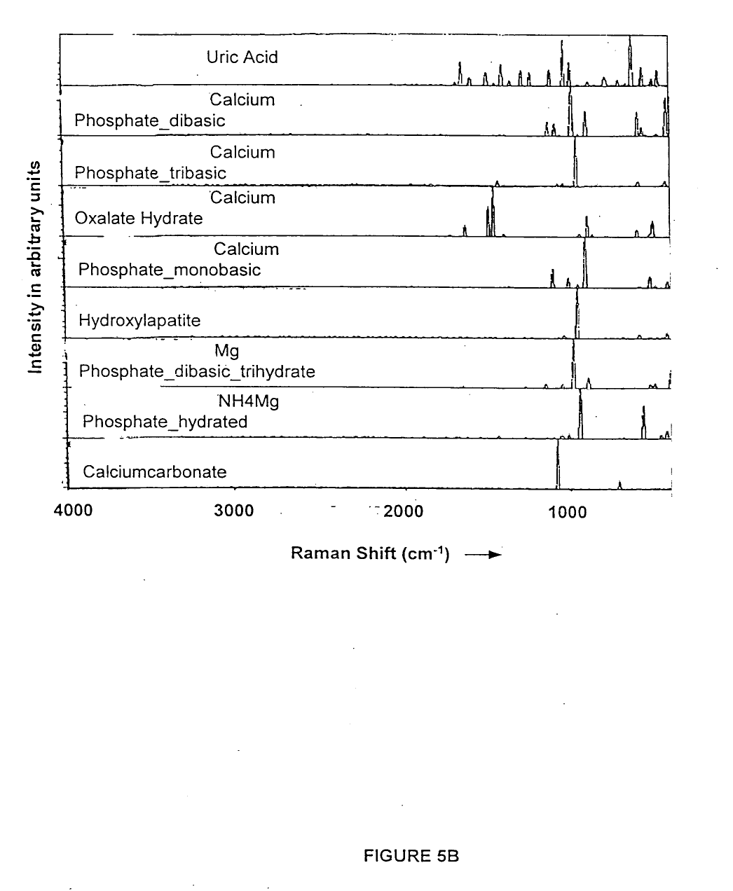

The present invention uses Raman spectroscopy to generate rapid and non-destructive fingerprints of substances that reflect not only the chemical identity, but also the physical state. Raman spectra, although technically challenging, may reflect the effect of interactions with other molecules for identifying various forms of substances. The use of Raman spectroscopy in the methods of the invention preferably requires control and suppression of noise and interference. Despite the weak Raman signal, the large fluorescence due to the solvent, and/or surroundings, and/or impurities, which make consistent detection of many Raman emissions difficult, the invention provides methods to overcome these difficulties. Thus, in FIG. 2, during step 245 the estimate of the composition of crystals may be made by Raman spectroscopy. Example Raman spectra for various crystals comprising calcium oxalate are shown in FIGS. 5A and 5B at different resolutions. An examination of the spectra indicates that the presence of different components in the medium changes the Raman signature, thus allowing for in-situ compositional analysis. A significantly more accurate method, described later, for compositional analysis is the analysis of Raman spectra by binning it. This method is fast and accurate as is required for a high throughput application. Binning is able to reliably identify the bin for a sample in real time, i.e., on the order of minutes to less than a minute and even as short a time interval as a few milliseconds. Preferably, the binning procedure is implemented to bin a sample in less than a thousand milliseconds, more preferably in less than a hundred milliseconds, and even more preferably in less than ten milliseconds. Of course, even faster times are possible with faster computers. Next some improvements in and limitations of Raman spectroscopy are discussed in the context of the various embodiments of the invention.

Many techniques have been developed for obtaining acceptable Raman spectra as is briefly described next. It should be noted that this description emphasizes improvements in Raman spectroscopy by way of illustration and not as a limitation on the scope of the claimed invention.

An improvement in reproducibly obtaining Raman spectra for samples of interest requires sensitive detection and specificity for rejecting non-Raman “noise.” The strength of Raman emissions is improved by the use of lasers to excite the target substance. Use of a carefully selected wavelength also results in Resonant Raman spectra. Sample preparation techniques resulting in adsorbing of a target to a surface further increase Raman signals, although such preparation is not always possible or even desirable in the case of in-situ data collection. Of course, optical amplifiers further improve sensitivity and specificity. Each of these techniques or process steps can be used alone or in combination within the methods of the invention.

Filtering techniques to be used within the invention for rejecting noise include but are not limited to temporal, spatial, and frequency domain filtering. Spatial filtering requires collecting emissions from a target in the focus of an objective to reject noise from surrounding sources. Such confocal techniques allow scanning of a target to reduce unwanted noise due to emissions from the material surrounding the target area.

Temporal filtering which rejects or accepts signals received in a particular time window can also be used within the methods of the invention. In the case of Raman spectra, temporal filtering relies on the different times taken for emission of Raman spectra and the background fluorescence spectra or on collecting sufficient data to meet a prescribed S/N threshold. Notably, Raman emissions, while weak, are detectable much earlier than fluorescence following excitation. Furthermore, fluorescent radiation continues over a significantly longer period, thus making possible selection of time windows for collecting Raman signal with a higher S/N ratio than otherwise. An example of such filtering is provided by Matsousek et al. in “Fluorescence suppression in resonance Raman spectroscopy using a high-performance Picosecond Kerr Gate,” in J. Raman Spectroscopy, vol. 32, pages 983-988 (2001). The Kerr gate realized by Matousek et al. exhibits a response time of about 4 picoseconds, thus allowing collection of Raman emissions during a window of 4 picoseconds following an exciting laser pulse. This example should be regarded as illustrative and not limiting as to temporal considerations in collecting and filtering spectra in possible embodiments since other gates, including virtual gating techniques are also intended to be within the scope of the claimed invention.

These filtering techniques can be augmented with mathematical filtering, e.g., convolution with the characteristic shape of a Raman line to further reduce the noise and reject unwanted frequencies and emissions. Notably, all of the described filtering techniques may not be employed in the same setup or within the same method.

This invention encompasses a method of determining an unknown state of a substance by examining it spectroscopically, in-situ if required, and processing an acquired spectrum to determine the location of a plurality of peaks. The location of these peaks is compared to the locations of the peaks in the acquired spectrum to a plurality of spectra using a similarity metric. Some examples of similarity metrics include the Tanimoto coefficient, Dice coefficient, Tversky index, Euclidean distance, and Hamming distance. This comparison results in assigning the acquired spectrum to one of a plurality of bins. The unknown state corresponding to the acquired spectrum is then identified as the state corresponding to the bin of the acquired spectrum. In another aspect, pair-wise comparisons between spectra, in accordance with a selected metric, from a plurality of spectra may be clustered by hierarchical clustering, or other clustering techniques to generate the aforementioned bins.

It should be noted that the described method does not require the presence of reference states. It can analyze spectral data with no knowledge of underlying states to generate a set of bins. Thus, the generation of a bin may instead signal the presence of a state or solid form, including a possibly previously unknown state or solid form, which may be useful in its own right or as a reference state for other applications. In effect, the method builds upon the binning of Raman and other spectra in accordance with their structure.

Ultraviolet spectroscopy is very useful for studying the rates of solid-state physical changes. Such studies require that the amount of reactant or product be measured quantitatively. Pendergrass et al. (1974) developed an ultraviolet method for the analysis of the solid-state thermal physical change of azotribenzoylmethane. In this reaction, the yellow form (H1) thermally rearranges to the red (H2) and white (H3) forms in the solid state. All three compounds (H1, H2, and H3) have different chromophores, so that this reaction is amenable to analysis by ultraviolet spectroscopy. Pendergrass developed a matrix-algebra method for analyzing multi component mixtures by ultraviolet spectroscopy and used it to analyze the rate of the solid-state reaction under various conditions.

The observation of NMR spectra requires that the sample be placed in a magnetic field where the normally degenerate nuclear energy levels are split. The energy of transition between these levels is then measured. In general, the proton magnetic resonance spectra are measured for quantitative analysis, although the spectra of other nuclei are also sometimes measured.

There are three important quantities measured in NMR spectroscopy: the chemical shift; the spin-spin coupling constant, and the area of the peak. The chemical shift is related to the energy of the transition between nuclei, the spin-spin coupling constant is related to the magnetic interaction between nuclei, and the area of the peak is related to the number of nuclei responsible for the peak. It is the area of the peak that is of interest in quantitative NMR analysis.

The ratio of the areas of the various peaks in proton NMR spectroscopy is equal to the ratio of protons responsible for these peaks. For multi component mixtures, the ratios of areas of peaks from each component are proportional both to the number of protons responsible for the peak and to the amount of the component. Thus, the addition of a known concentration of an internal standard allows the determination of the concentrations of the species present. For cases where the ratio of starting material and product is desired it is not necessary to add an internal standard.

Polarography is a special type of voltammetry that uses a dropping mercury electrode. In this experiment an electrochemical reaction is allowed to proceed at a given potential at the electrode and the current flow is measured. The current flow is proportional to the amount of species present. This proportionality is reflected in the well-known Ilkovic equation. Since different compounds undergo reactions at different potentials, polarography, at least in favorable cases, allows the quantitative analysis of one species in the presence of others. However, the “resolution” of polarography is significantly less than other methods such as ultraviolet or NMR spectroscopy. This is because members of one functional class of compounds (i.e., the substituted quinones) undergo electrochemical reaction at potentials close enough that significant overlap between their polarograms occurs. Polarography is sensitive to concentrations down to 10⁻⁵ to 10⁻⁶ M, depending on the functional group undergoing electrochemical reaction.

The polarographic experiment must be performed in the absence of dissolved oxygen. This is because oxygen reduction will produce a wave that obscures the reduction of most materials of interest. With water solutions, 5 min of nitrogen bubbling is usually sufficient to remove dissolved oxygen; however, organic solvents often require longer bubbling.

High-pressure liquid chromatography is probably the most widely used analytical method in the pharmaceutical industry. It is also of utility in fractionating or otherwise preparing substances from biological fluids including urine for evaluating substances that play a role or may play a role in the development, prevention, or treatment of nephrolithiasis.

In some ways, a high-pressure liquid chromatography resembles a gas chromatography in that it has an injector, a column, and a detector. However, in high-pressure liquid chromatography it is not necessary to heat the column or sample, making this technique useful for the analysis of heat sensitive materials. In addition, a wide range of column materials is available, ranging from silica to the so-called reversed-phase columns (which are effectively nonpolar columns). As with gas chromatography, several detectors are available. The variable-wavelength ultraviolet detector is particularly useful for pharmaceuticals and for studying the solid-state reactions of drugs, since most drugs and their reaction products absorb in the ultraviolet range. In addition, extremely sensitive fluorescence and electrochemical detectors are also available.

A typical analysis by HPLC proceeds in the following manner:

Step 1. Selection of column and detector—these selections are usually based on the physical properties of the reactant and the product.

Step 2. Optimization of flow rate and column length to obtain the best separation.

Step 3. Analysis of known mixtures of reactant and product and construction of a calibration curve.

Thin-layer chromatography (TLC) provides a very simple and efficient method of separation. Only minimal equipment is required for TLC, and very good separations can often be achieved. In general, it is difficult to quantitate TLC, so it is usually used as a method for separation of compounds.

A typical investigation of a solid-state reaction with TLC proceeds as follows:

Step 1. The adsorbent (stationery phase) is selected and plates either purchased or prepared. Usually silica gel or alumina is used.

Step 2. The sample and controls, such as unreacted starting material, are spotted near the bottom of the plate and developed in several solvents until the best separation is discovered.

This procedure then gives the researcher a good idea of the number of products formed. Based on these preliminary studies, an efficient preparative separation of the products and reactant can often be designed and carried out.

The vision station is similar to that described in patent applications (and parent applications thereof) including U.S. patent application Ser. No. 09/756,092 filed on Jan. 8, 2001, U.S. provisional patent application No. 60/318,138 filed on Sep. 7, 2001 and the PCT patent application No. PCT/US01/00531 filed on Jan. 8, 2001, all of which are incorporated herein by reference in their entirety.

In the context of the present invention a suitable vision station allows for the analysis of solid forms in high-throughput arrays, such as presented by a multiwell plate or a tube and block apparatus. In particular, the vision station enables examination of a collection of crystals or precipitates comprising calcium oxalate and formed or grown from media containing calcium oxalate. The vision station also allows examination of a single crystal or deposit comprising calcium oxalate. The examination includes detection and measurement of birefringence by examining samples in polarized light, turbidity, form and habit of crystals, and the like. Of course, the vision station is useful for detecting and evaluating the absence of calcium oxalate comprising calculi, crystals, or precipitates that cause kidney disease or disorders.

The vision station has various other facilities, such as a light source and a camera. A suitable camera can be any unit capable of yielding photographic images of the contents of containers, e.g., the presence or absence of solids or solid forms in a tube, but is preferably one capable of digital capture. In a preferred embodiment of the present invention, a CCD (charge coupled device) camera provides adequate sensitivity, but other digital capture devices may also be used. The light source is selected based on the types of containers being used and the design of the experiment, and by way of example, the light source may be visible light, laser light of varying wavelengths, laser monochrome, plane-polarized, or circularly polarized light. In an example embodiment, the light source is white light from one or more tungsten lamps. Depending on the mode of application of the vision station, light can be brought in from the top of the array, the bottom, or from the side. The side approach is dependent on the ability to expose the container contents generated in the arrays above or below the array when viewed end on. Blocks containing removable containers allow improved access by light to the sample due to the ability to elevate the containers from the block, either by hand, or using an automated means.

In experiments to generate, discover, or identify samples or compounds-of-interest, samples are generally subjected to some form of analysis, including without limitation, Raman spectrometers, IR spectrometers, NIR spectrometers, fluorimeter, NMR spectrometers, and the like in the course of determining the sample's physical form.

With use of color images, one can capture polychromism (i.e., multi-color crystals) information from the experiment with a suitable camera, or simply run the analysis with black and white images and look for bright pixels. In addition, a quarter-wave retarding filter can be used to confirm the presence of crystals by causing a color shift when the filter is applied.

In a preferred embodiment, the invention is useful to identify, discover or optimize conditions, compounds, or compositions that prevent, inhibit, or modify crystallization, precipitation, formation, growth, agglomeration, modification, or deposition of calcium oxalate calculi. Possible changes include changes in the size or habit of calcium oxalate crystals, or in a propensity to aggregate or polymerize for macromolecular aggregates comprising calcium oxalate. In this embodiment, an array is prepared comprising samples having a medium comprising calcium oxalate, preferably at super saturating levels, to induce formation of a precipitate or crystal in a manner similar to that in kidney diseases or disorders. This formation may be in the presence of cells in cell culture or of tissue/organ culture, including culturing by perfusion of suitable fluids. Thus, the contents of one or more of the samples is prepared to mimic the in-vivo conditions suitable for forming a crystal or calculus in the kidney, bladder, urethra, tubules, and the like. In addition, formed crystals can be placed in contact with cells in culture or organs/tissues in culture. This enables evaluation of adhesion of the precipitate or crystal to the cells/tissue/organ surface and/or migration with respect thereto. In addition, if desired, various components in varying concentrations are added to selected samples and the samples are processed. If desired, particular samples can be processed under various conditions. Preferably, one or more of the samples differs from one or more other samples by:
(i) the presence or an amount of dissolved calcium oxalate;
(ii) the identity or an amount of the medium;
(iii) the identity or an amount of at least one of the components, preferably a test compound; or
(iv) the pH.

For example, samples can have one or more of the following components at various concentrations: compounds and compositions that prevent, inhibit, or modify precipitation, formation, crystallization, nucleation, and/or growth; nucleation promoters; compositions or compounds that affect crystal habit; nutrients; pharmaceuticals; nutraceuticals or components thereof; herbal preparations or components thereof; hormones; steroids; proteins and peptides; chelating agents; excipients; organic solvents; salts; acids; bases; gases; or stabilizers.

Preferably, one or more negative controls contains only the medium and components necessary to mimic the in-vivo conditions suitable to form a calcium oxalate crystal or calculus and that does generate the calcium oxalate crystals, thus to guaranteeing the integrity of the experiment. After processing, the samples can be analyzed to identify those samples having crystals or deposits and those that do not. The samples that do not have calcium oxalate crystals/precipitates are predicative of conditions, compounds, or compositions that prevent, inhibit, or modify crystallization, precipitation, formation, growth, or deposition of calcium oxalate calculi.

The positive sample can be further analyzed to determine the calcium oxalate calculus' structural, physical, pharmacological, or chemical properties. Structural properties include whether the solid is crystalline or amorphous, and if crystalline, the polymorphic form and a description of the crystal habit. The calcium oxalate calculus' composition can be analyzed to determine whether it is a hydrate, solvate, or a salt. Also, the surface-to-volume ratio and the degree of particle agglomeration can be determined. Other physical properties that can be measured include melting point, solubility, strength, hardness, compressibility, compactability, and resistance to energy forms, such as ultrasound, shock waves, and laser energy. Thus, the experiment can reveal conditions, compounds, or compositions that form calcium oxalate calculi more readily or that form calcium oxalate that are more readily fragmented or destroyed than would otherwise be formed (e.g., those of the negative control or those formed in vivo). For example, compounds, compositions, or conditions that induce formation of calcium oxalate calculi that are easily destroyed or fragmented by ultrasound or shock waves are therapeutically exploitable.

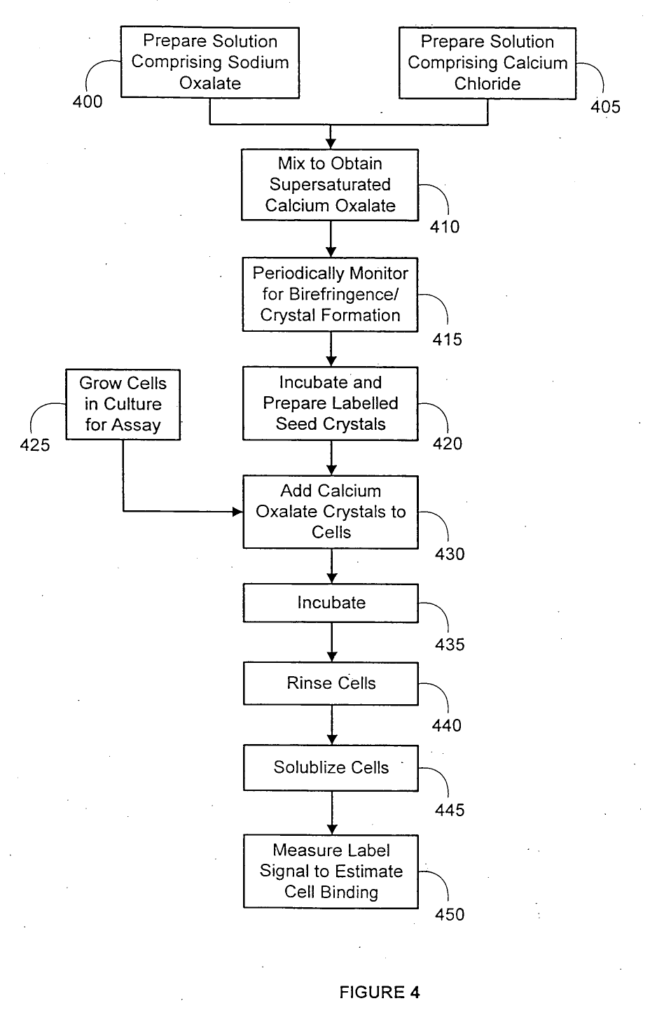

FIG. 4 illustrates an embodiment for screening for components that may modify the adhesion, and possibly the migration properties of calcium oxalate crystals. During steps 300 and 305 solutions comprising sodium oxalate and calcium chloride respectively are prepared as described in FIG. 1 followed by mixing during step 410. The solution comprising sodium oxalate mixture also has fluorescein, although other fluorophores may be used in addition or instead of fluorescein. Monitoring during step 425 results in the detection of crystals that are labeled with a fluorophore. The labeled crystals are washed and prepared during step 420 for exposure to cells.

During step 425 cells, any suitable renal epithelial cell line (or cells) such as MDCK (American Type Culture Commission (“ATCC”) Number CCL-34), LLC-PK₁ (ATCC CL-101), mIMCD (ATCC CRL-2123), and the like are grown into a monolayer. The cells are incubated at 37° C. in a 5% CO₂ atmosphere in Dulbecco's Minimal Essential Media (hereinafter “DMEM”) supplemented with 10% fetal bovine serum. The substrate may be glass or plastic or permeable membrane filters or other supports. The cells are refed as necessary to achieve suitable density and confluence, although this is not a requirement for practicing the invention. Thus with the use of multiwell plates or other plates several cell layer preparations are possible for screening in an array format. It should be noted that although the described procedures are for cell lines, similar processing is known for establishing or even temporarily maintaining tissue such as renal tubules in culture for various durations.

Following growth, the cells are washed and the prepared labeled crystals of calcium oxalate added during step 430. During step 435 the crystals are incubated with the cells in medium comprising test components for modifying adhesion or mobility of crystals. Such components include but are not limited to proteins, peptides, drugs, and the like.

Following incubation the cells are rinsed with physiological saline or a similar buffer during step 440 to remove weakly or non-adherent calcium oxalate crystals and the cells solubilized during step 445. Suitable solubilization buffers contain detergents such as sodium dodecyl sulfate (“SDS”) at sufficient concentration to solubilize the cell but not to interfere with the assay. A preferred concentration of SDS is about 0.2%.

The fluorescence intensity of the solubilized cell material provides a relative measure of adhesion/migration of calcium oxalate crystals to/on the cells, thus providing a method for high throughput measurement of modification of adhesion and migration related properties.

Usually, after the methods discussed herein are completed, one may select and further analyze samples with the most desirable properties, for example, selection and clinical exploitation of samples wherein crystallization, precipitation, formation, modification, or deposition of calcium oxalate calculi/crystals was prevented or inhibited, relative to a negative control; or selection and clinical exploitation of samples where formation of a desirable substance was promoted, relative to a negative control. From such samples, clinically-relevant conditions, compounds, or compositions can be identified and exploited to treat (e.g., reverse) or prevent the disease itself, the cause of the disease, or the symptoms of the disease.

Clinically relevant compounds and compositions can be assayed for use as drugs to treat or prevent the diseases of physical-state transition mentioned above. For example, the clinically relevant compounds and compositions can be assayed for effectiveness, potency, toxicity, absorption, and metabolism using in-vitro and in-vivo tests and experiments well known to those skilled in the pharmaceutical arts.

The methods of the invention have already been used to identify several compounds that have utility in either or both the in vitro screening methods and/or the treatment of nephrolithiasis in mammals. The compounds screened were selected based upon the types of functional groups present and the positions of functional groups.

Thus, the invention encompasses the use of one or more compounds selected from the group consisting of 4-bromomandelic acid, sucrose octaacetate, agaric acid, tetrahydrofuran-2,3,4,5-tetracarboxylic acid, atrolactic acid hemihydrate, methylene blue, lactobionic acid, benzilic acid, mandelic acid, lactobionic acid hemi-calcium salt, indomethacin, furosemide, hippuric acid, probenicid, aromatic compounds, heteroaromatic compounds, quazalones, perazanones, uricils, statins, sulfated alcohols, alkylsulfides, ethers, polyethers, peptoids, and sugars, and salts, hydrates, solvates, esters, or prodrugs thereof, to treat kidney disease.

The invention further encompasses the use of urine from a patient to diagnose whether a disease, condition, or disorder associated with calcium oxalate deposits or calculi (e.g., nephrolithiasis) is present or to determine the risk of experiencing such disease, condition, or disorder. This urine can be subjected to a screen for nucleation, crystallization, precipitation, crystal growth, agglomeration, etc. of calcium oxalate crystals/calculi. Alternatively, the patients can be screened to determine if their urine responds well to a particular therapeutic so that the patient is matched with the best treatment protocol.

The compounds used within the methods of the invention are known and/or are commercially available. Thus, they can be purchased or synthesized according to standard organic synthetic methodologies known in the art.

The method and system described above are useful for kidney diseases and disorders as well as many other diseases and disorders. In particular, preparation of supersaturated plasma preparation of calcium phosphate or calcium oxalate may be similar with optical monitoring of crystallization or precipitation.

An exemplary method for high throughput screening to identify potential inhibitors of nucleation, growth, agglomeration, aggregation, adhesion, or migration comprises dispensing a solution having supersaturated calcium oxalate into an array, to test the effect of conditions/compounds added to various samples in the array. The array is incubated at a predetermined temperature with an assessment of changes in the solution by techniques such as measuring turbidity, microscopic morphometry, measuring total light output, measuring the spatial heterogeneity of light output, image processing, particle counting, and birefringence.

Preferably, the solution comprising supersaturated calcium oxalate comprises urine such as synthetic urine or human urine. The incubation temperature is preferably, not as a requirement, 37° C. As discussed previously, the disclosed techniques are flexible and can easily handle arrays having 24, 48, 96, 384, 1536, 10,000, 100,000 or more samples. These samples may be held in multiwell plates, including plates having glass-bottomed wells, or in a collection of containers in one or more blocks to allow for ease of manipulation and tracking of barcoded blocks. The containers may be rearrayed if desired with individual containers tracked by their respective location and the block barcode.

Preferably, calcium oxalate crystals are also used to seed the supersaturated calcium oxalate solution in some samples to assess their growth or the lack thereof. Absorbance at about 620 nm provides a measure of turbidity and a decrease in turbidity indicates either aggregation or crystals/precipitate or reduction in the precipitation/crystal growth of calcium oxalate crystals. It should be noted that turbidity may be measured at other wavelengths as well including wavelengths corresponding to visible light and ultraviolet light. Preferably, but not as a requirement, turbidity is measured by evaluating light scattered at an angle to the incident light. Preferably, such an angle is 90°, although other angles may be employed instead or in addition. Conditions resulting in reduction of precipitation/crystal growth of calcium oxalate crystals are thereby detected for further characterization of the samples and conditions responsible therefor.

In another aspect, a time course of the absorbance reveals a decrease in the absorbance relative to a control sample to identify a corresponding compound or condition useful for treating nephrolithiasis. Moreover, images exhibiting birefringence in polarized light allow for automated image analysis to detect formation of crystals as well as the number of crystals derived from the spatial heterogeneity of light output intensity.

In another aspect individual crystals are imaged, preferably in plastic- or glass-bottomed multiwell plates or individual glass tubes, to discriminate between nucleation, growth and agglomeration of crystals. Following incubation for a defined duration, with removal of solution to leave behind seed crystals, fresh supersaturated calcium oxalate solution is added to study crystal growth and/or aggregation. Alternatively, collecting and drying the crystals for use as seed crystals, e.g., as measured by a hemocytometer, also allows tracking of their growth/aggregation. Advantageously, the location and area of multiple crystals in a well is determined at various time points to obtain a measure of crystal growth both in the particular and in the aggregate for a given well and condition. To measure crystal aggregation, crystals in suspension are agitated at a specified speed for a predetermined period. Particle size is then compared prior to and following the agitation period.

Thus, the invention encompasses high-throughput assays for identifying compounds, especially small molecules, that can inhibit the formation and/or growth of calcium containing crystals and other types of crystals that otherwise result in a disease state, including kidney diseases or disorders. Such compounds may be known compounds or novel compounds and may act to prevent or alleviate one or more disease symptoms.

### 5.4. EXAMPLES

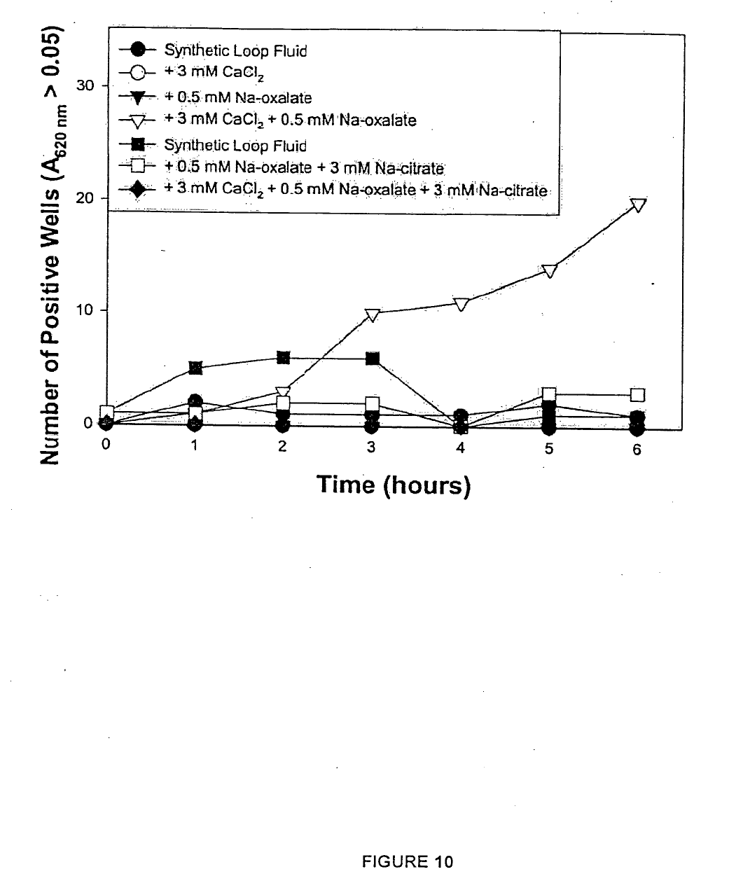

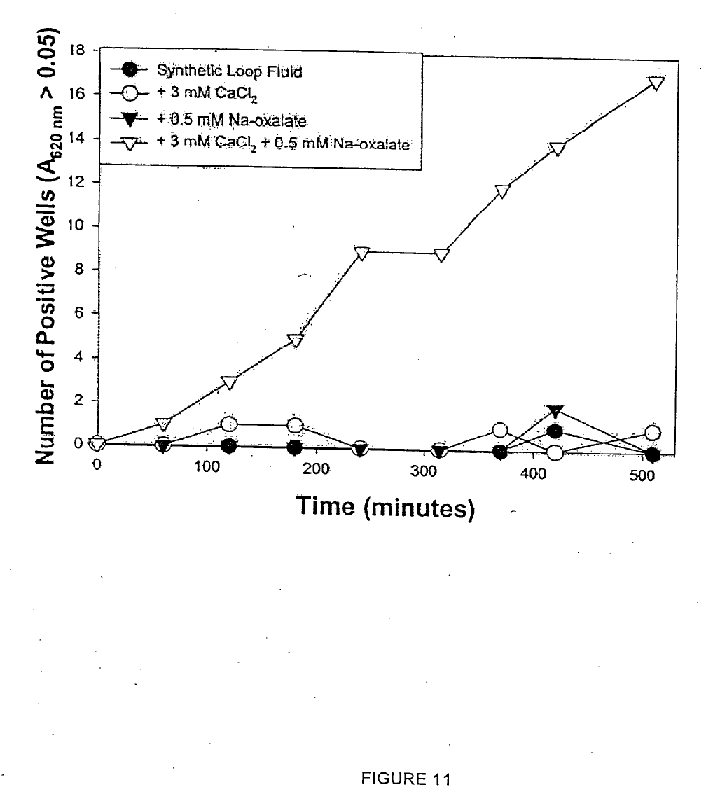

The disclosed system and method reveal that the known treatment for nephrolithiasis in administering citrate is effective, although limited, in reducing the formation of calcium oxalate crystals in a supersaturated calcium oxalate solution. This is shown in FIG. 10 and FIG. 11. As shown, the presence of 3 mM sodium citrate in supersaturated calcium oxalate (shown by closed diamonds) dramatically reduces the turbidity compared to supersaturated calcium oxalate by itself (shown by open triangles as in FIG. 10 and FIG. 11). The other curves are controls and include the effect of loop fluid, which comprises 260 mM NaCl, 21 mM KCl, 3 mM MgCl2, 3.33 mM NaH₂PO₄, 1.67 mM Na₂HPO₄, pH=6.5 (calculated), and has a pH=6.3 (measured). Not surprisingly, administration of citrate is one of the treatments for kidney stones in addition to the recommendation for plenty of fluids. However, discovery of additional components effective at lower doses than citrate or optimizing the dosing of citrate is made possible by the high throughput screening in the various embodiments of the invention.

In addition, various other components have been screened by the described procedures. In particular several compounds have been found to exhibit desirable properties in modifying or otherwise affecting calcium oxalate crystal growth. A non-exhaustive list includes 4-bromomandelic acid, sucrose octaacetate, agaric acid, tetrahydrofuran-2,3,4,5-tetracarboxylic acid, atrolactic acid hemihydrate, methylene blue, lactobionic acid, benzilic acid, mandelic acid, lactobionic acid hemi calcium salt, indomethacin, furosemide, hippuric acid, and probenicid. The molecular weights, amounts and concentrations used, and structures for some of the tested compounds are presented in FIGS. 9A and 9B. Some of the salient observations include the absence of plate like structures with the use of 4-bromomandelic acid but with larger sized crystals being formed; an increased number (countable) of crystals with sucrose octaacetate; formation of an emulsion/suspension by agaric acid, reduced number of intermediate to large crystals and increased number of small crystals with tetrahydrofuran-2,3,4,5-tetracarboxylic acid. A reduction in large crystals is seen with atrolactic acid hemihydrate and methylene blue (but difficulty in visualizing crystals); many small and large crystals with lactobionic acid; increasing number of small crystals with increasing levels of benzilic acid; presence of very large crystals in some wells with the use of lactobionic acid hemi calcium salt; reduction in the number of large crystals with little change in the number of small crystals with the use of indomethacin; furosemide resulted in many more large crystals; while hippuric acid and mandelic acid had little effect along with probenicid, which seemed to have a few more small crystals. Thus, the results are diverse and reflect many combinations of nucleation, growth/habit modifications that are pharmaceutically interesting.

Although the present invention has described in considerable detail with reference to certain preferred embodiments, other embodiments are possible. Therefore, the spirit and scope of the appended claims should not be limited to the description of the preferred embodiments contained herein. Modifications and variations of the invention described herein will be obvious to those skilled in the art from the foregoing detailed description and such modifications and variations are intended to come within the scope of the appended claims.

A number of references have been cited, the entire disclosures of which are incorporated herein by reference.

1. A method for identifying conditions, compounds, or compositions for modifying a crystal or a calculus comprising calcium oxalate, the method comprising:
preparing seed crystals of calcium oxalate;
incubating the said seed crystals in a supersaturated calcium oxalate medium comprising at least one of a plurality of components;
detecting a first crystal property; and
identifying a component corresponding to a change in said crystal property.

2-31. (canceled)

32. The method of claim 1, further comprising culturing kidney epithelial cells.

33. The method of claim 32, wherein the cultured cells are selected from the group consisting of: MDCK, LLC-PK₁, mIMCD, and tubule fragments.

34. The method of claim 32, further comprising preparing labeled calcium oxalate crystals.

35. The method of claim 32, further comprising incubating labeled calcium oxalate crystals with cultured kidney epithelial cells.

36. The method of claim 35, further comprising solubilizing washed cells following incubation with labeled calcium oxalate crystals; and measuring a signal corresponding to a label in the labeled calcium oxalate crystals.

37-102. (canceled)
